(19) United States
(12) Patent Application Publication
(10) Pub. No.: US 2024/0247053 A1
(43) Pub. Date: Jul. 25, 2024

(54) COMBINATIONS FOR ALLERGY THERAPY

(71) Applicant: IgGenix, Inc., South San Francisco, CA (US)

(72) Inventors: Derek Croote, San Mateo, CA (US); Joyce Wong, South San Francisco, CA (US)

(21) Appl. No.: 18/415,554

(22) Filed: Jan. 17, 2024

Related U.S. Application Data
(63) Continuation of application No. 17/980,397, filed on Nov. 3, 2022, now abandoned.
(60) Provisional application No. 63/287,190, filed on Dec. 8, 2021.

(51) Int. Cl. C07K 16/18; A61P 37/08; C07K 16/16

(52) U.S. Cl. CPC C07K 16/18; A61P 37/08; C07K 16/16

(57) ABSTRACT

A method of inhibiting anaphylaxis includes administering to a subject a composition comprising a first antibody, or fragment thereof, that binds to an allergen and a second antibody, or fragment thereof, that binds to the allergen, wherein the first antibody and the second antibody are from selected, epitope bins for an allergen that show the highest relative efficacy.

### TECHNICAL FIELD

The disclosure relates to the treatment of allergies.

### BACKGROUND

Allergy is estimated to affect 30% of the global population. The prevalence of diagnosed allergies is continually increasing due to numerous factors, but in part due to changes in environmental exposures, the recognition of new allergens and allergic responses, as well as the increased availability of allergy tests.

Allergies are characterized by a number of conditions caused by hypersensitivity of the immune system to otherwise harmless substances in the environment. In general, an allergic reaction occurs when components of the immune system overreact to the presence of a substance (an allergen) that, absent the allergy, would not cause a reaction. Allergies have a negative impact on individuals' quality of life and can lead to societal and personal economic burdens. As mere exposure to certain allergens can have life-threatening consequences, people suffering from allergies are often required to be hypervigilant and forced to alter their behavior to avoid allergens.

As generally understood, an allergen is a type of antigen that produces an abnormally vigorous immune response in which the immune system combats a perceived threat that would otherwise be harmless. In technical terms, an allergen is an antigen that is capable of stimulating a type I hypersensitivity reaction mediated by Immunoglobulin E (IgE) in atopic individuals. Most humans mount significant IgE responses only as a defense against parasitic infections. However, some individuals may respond to common environmental antigens. This predisposition is called atopy. In atopic individuals, non-parasitic antigens stimulate inappropriate IgE production, leading to type I hypersensitivity.

Emerging research has shown that allergies are not homogenous conditions. Allergy research has shown that many common allergies are more complex than previously thought. For example, patients with peanut allergies may be allergic to one or more proteins found in peanuts as well as one or more epitopes of said proteins. Further, people with a particular allergy can produce different IgE antibodies in response to the same antigen.

Given the complex nature of allergies, discovering and developing therapeutics requires screening potential treatments across a broad range of targets, including across the multiple epitopes of an allergen. Quickly ascertaining the most promising epitopes to target with a potential allergy therapeutic not only reduces the resources required for therapeutic development, but also leads to more effective treatments.

### SUMMARY

The invention provides methods for developing allergy treatments by selecting a subset of allergen epitopes as therapeutic targets, while discarding other epitopes as potential targets for therapeutic development. Thus, the resulting treatments and compositions, which are also included as part of the present invention, include proteins that bind to a subset of allergen epitopes, without binding to every available epitope. Despite binding to only a subset of epitopes, the compositions of the invention effectively block the allergen from binding to IgE antibodies, thus blocking the allergic response.

In one aspect, the invention provides methods for treating allergy by administering compositions that include proteins that bind to more than one place on an allergen, without needing to bind to every available epitope, and effectively block the allergen from binding to IgE antibodies, thus blocking the allergic response. The antibodies implicated in allergy bind to a specific part of the allergen. The region of an allergen recognized by an antibody is known as the epitope of that allergen for that antibody. Any given allergen may have three or more epitopes, and the disclosure provides results showing that compositions comprising antibodies that bind to two epitopes are effective at blocking IgE binding. In fact, even for an allergen with three or more (e.g., five) epitopes, results presented here show that combinations of therapeutic antibodies that bind to two of the three or more epitopes are essentially as good as combinations that target a greater number (e.g., all five). Thus, the invention provides methods and compositions that use first and second antibodies in combination to block IgE binding to the allergen, where the combination of two works substantially as well as combinations comprising antibodies that bind to three or more epitopes of the allergen. An insight of the invention is that for multi-epitopes allergens, there is a principle of diminishing returns. Where different antibodies (or fragments thereof, or other epitope binding molecules) independently target different epitopes of an allergen, two antibodies show better IgE blocking (e.g., in in vitro blocking assays) than the sum of each antibody measured independently. But also, the two antibodies in combination do as well as three or more independent antibodies, and provide substantially the full blocking effect of a combination that includes three or more independent antibodies. Methods and compositions of the invention include two or more therapeutic proteins, such as monoclonal antibodies, that bind to two more respective epitopes on the allergen. Results herein show that binding to two or more epitope from different parts of the allergen offers significantly better blocking of allergen-IgE binding that offered by any single monoclonal antibody and better than a sum of blocking of each antibody measured in vitro.

Additionally, methods of the invention are useful to map therapeutic proteins, such as monoclonal antibodies, to their cognate epitopes on an allergen. The mapping is useful to identify the number of available epitopes of an allergen. For example, methods of the invention reveal that an important peanut allergen has at least five distinct epitopes. Using that insight, compositions are provided that include therapeutic proteins that bind to any or all of the epitopes on an allergen to block IgE binding. However, even for an allergen with five available epitopes, results presented here show that it may be sufficient to include just two therapeutic proteins. In the peanut example, a composition may include five different therapeutic proteins (e.g., five different monoclonal IgG4 antibodies) that each bind to one of the five available epitopes, but need only include two for a substantially similar quantitative result. Noting that methods of the invention are useful for epitope mapping to discover a number of available epitopes, the invention provides compositions that include multiple therapeutic proteins that bind to number of available epitopes on an allergen. For example, a different allergen may be subject to epitope mapping and discovered to have seven distinct epitopes, and a composition may be provided that includes at least one therapeutic protein for each of the seven epitopes, but preferably includes two antibodies, specific for a respective two of the seven epitopes, despite the potential availability of seven epitopes. Targeting two will do substantially as well as targeting any number of epitopes from three to seven.

Methods of the invention also provide compositions that include therapeutic proteins that bind to multiple distinct epitopes. Epitope mapping methods of the disclosure reveal certain therapeutic proteins to readily bind to one of multiple different epitopes. For example, with the peanut allergen discussed above, epitope mapping/binning was performed to identify at least five epitope bins on the allergen. The mapping data, based on a blocking assay, reveal in that case that proteins of bin 3 exhibit useful promiscuity and may be used to bind to epitopes of bin 1, bin 2, bin 4, and bin 5, as well as Bin 3. Noting the methods of the invention are to discover proteins that bind to a number of available epitopes, the invention provides compositions that include a therapeutic protein that binds a number of available epitopes on an allergen. The promiscuous therapeutic protein can be used alone to bind and block multiple epitopes or may be used in combination with other therapeutic proteins.

Methods and compositions of the invention use measurements of the ability of one isolated antibody to compete with and block another isolated antibody from binding to an allergen. When multiple unique isolated antibodies, e.g., candidate monoclonal antibodies, are tested for their ability to block one another from binding to the allergen, the test results reveal identifiable groups of antibodies, within which groups each antibody blocks the other group members from binding the allergen. An insight of the invention is that those groups of antibodies can be classified as distinct bins, dubbed epitope bins, within which member antibodies recognize overlapping epitopes on the allergen.

The ability of an antibody to block other antibodies can be measured in vitro by a competitive binding assay, such as a blocking ELISA. Groups of antibodies that block one another from binding the allergen are said to define an epitope bin. Competitive binding assays reveal that allergens may have several distinct and well-defined epitope bins.

Such competitive binding assays may be used, in turn, to measure the ability of a candidate monoclonal antibody to block a patient IgE antibody from binding to an allergen. It may be theorized that a candidate monoclonal antibody could be used therapeutically, and administered to a patient. If the therapeutic antibody blocks IgE from binding to allergen in the patient serum, the therapeutic antibody could block or inhibit the allergic response and potentially prevent anaphylaxis. The ability to block IgE may be measured by an ex vivo competitive binding assay, such as a benchtop blocking ELISA with a colorimetric result. An object of such a test is to identify candidate therapeutic antibodies that prevent the colorimetric result from antigen-IgE binding in the blocking ELISA.

When multiple unique isolated antibodies, e.g., candidate monoclonal antibodies, are tested, each alone and in various combinations, for their ability to block antigen-IgE binding, a remarkable result obtains. Combinations of the candidate antibodies far outperform any single candidate antibody when the combination includes antibodies from any two epitope bins. The result is consistent; every tested combination representing two bins strongly outperforms each and every single antibody when tested alone.

That discovery is employed to provide methods of inhibiting anaphylaxis or treating allergy. Methods of the invention include administering a composition that includes a first antibody, or fragment thereof, that binds to an allergen and a second antibody, or fragment thereof, that binds to the allergen. The first antibody and the second antibody are selected to not block one another from binding to the allergen, and they thus have the effect of blocking IgE antibodies of the subject from binding to the allergen. Preferably the first antibody binds a first epitope of the allergen and the second antibody binds a second epitope of the allergen. The first epitope and the second epitope may be different portions, e.g., spatially distinct parts, of the allergen. The first antibody and second antibody may be selected so that the first epitope and the second epitope are from different “epitope bins”, where each epitope bin is defined to include antibodies that block each other from binding, e.g., as demonstrated in an in vitro assay such as an ELISA blocking assay. Such an in vitro assay may similarly show that each of the first epitope and the second epitope exhibits minimal cross-reactivity with the first antibody and the second antibody, i.e., the first epitope is exclusive to the first antibody and does not bind to, or cross-react with, the second antibody, while the second epitope is exclusive to the second antibody and does not bind to, or cross-react with, the first antibody.

For compositions used in methods of the invention, the first antibody and second antibody may be monoclonal antibodies. In preferred embodiments of the methods, the first antibody and/or second antibody are IgG antibodies. The first antibody and second antibody may be provided in an aqueous suspension comprising a pharmaceutically acceptable excipient or buffer.

In certain aspects, the invention provides a pharmaceutical composition for the prevention or treatment of allergy and/or the inhibition of anaphylaxis. The composition includes a first antibody, or fragment thereof, that binds to an allergen and a second antibody, or fragment thereof, that binds to the allergen wherein the first and second antibodies in combination block IgE binding to the allergen substantially as well as combinations comprising antibodies that bind to three or more epitopes of the allergen. The first antibody and the second antibody preferably do not block one another from binding to the allergen. Preferably, the first antibody binds a first epitope of the allergen and the second antibody binds a second epitope of the allergen. The first epitope and the second epitope may be formed by different portions of the allergen. The first antibody and second antibody may be selected so that the first epitope and the second epitope are from different epitope bins, where each epitope bin is defined to include antibodies that block each other from binding and/or the first epitope and the second epitope are selected for minimal cross-reactivity with the first antibody and the second antibody.

For compositions of the invention, the first antibody and second antibody may be monoclonal antibodies. In preferred embodiments, the first antibody and/or second antibody are IgG antibodies. The first antibody and second antibody may be provided in an aqueous suspension comprising a pharmaceutically acceptable excipient or buffer.

In embodiments for treating or inhibit peanut allergy, the allergen may be Ara h 2 and the first epitope and the second epitope may be different and each bind to an epitope on Ara h 2 that includes one of the following sets of residues of Ara h 2: 1-9; 10-18; 21-30; 31-39; 40-54; 55-60; 109-117; 121-130; and 137-146. In other embodiments, the allergen is from milk, egg, tree nut, fish, shellfish, soy, legume, seed, wheat, cat, birch, latex, pollen, or fungus.

Additionally, methods of the invention are useful to streamline the development of therapeutics targeting multiple epitopes. Using certain methods of the invention, each epitope of an allergen may be assayed for an early assessment of its potential as a target for developing a therapeutic, independent of other epitopes. Thus, while any given allergen may have three or more epitopes, the disclosure provides results showing that compositions comprising antibodies that bind to a select subset of the epitopes, e.g., one or two epitopes, are effective at blocking IgE binding. Methods of the invention include administering compositions of the invention, which include by way for example, a first antibody or fragment, that binds to a first epitope on an allergen having more than two epitopes and a second antibody or fragment thereof, that binds to a second epitope on the allergen, wherein each antibody/fragment are selected from two different epitope bins from which antibodies were identified as independently showing greater inhibition of allergen mediated activation in cells relative to antibodies from other epitope bins for the allergen. Concurrently, even between therapeutics that bind to the same number of epitopes, those that bind to a selected subset of epitopes will produce a superior therapeutic effect.

Accordingly, the present invention provides pharmaceutical compositions for allergy treatment. For example, such a composition may include, a first antibody, or fragment thereof, that binds to a first epitope on an allergen having more than two epitopes and a second antibody, or fragment thereof, that binds to a second epitope on the allergen. Such a composition may be developed using methods of the invention such that the first antibody and second antibody are selected from two different epitope bins from which antibodies were identified as independently showing greater inhibition of allergen mediated activation in cells relative to antibodies from other epitope bins for the allergen.

In certain aspects, each epitope bin is defined to include antibodies that block one another other from binding to the allergen. Antibodies from different epitope bins, therefore, do not block one another from binding to the allergen.

In some embodiments, the first and second epitopes are formed by different portions of the allergen. The first epitope and the second epitope may be selected as therapeutic targets because they show minimal cross-reactivity with the second antibody and the first antibody, respectively.

In certain compositions of the invention, the first antibody and second antibody are monoclonal antibodies. Alternatively or additionally, the first antibody and/or second antibody are IgG antibodies.

In certain aspects, the allergen treated by a composition of the invention originates from the list consisting of: peanut, milk, grass(es), tree(s), weed(s), venom(s), mold(s), egg, tree nut(s), fish, shellfish, soy, legume, seed(s), dust mite, animal dander or saliva, microorganism(s), and wheat.

In preferred aspects, the first and second antibodies in combination block IgE binding to the allergen substantially as well as combinations comprising antibodies that bind to three or more epitopes of the allergen.

Compositions of the invention include those in which the target cat allergen is Fel d 1, those in which the target is Ara h 2, and those in which the target is Ara h 6.

In an exemplary composition of the invention, the first and second antibodies are formulated together. In some aspects, the composition includes an anti-IgE antibody in an amount sufficient to reduce, but not eliminate, circulating IgE. In certain aspects, the first and second antibodies and the anti-IgE antibody are provided to the subject in separate dosage forms.

Methods and compositions of the invention may use assays that measure the ability of one isolated antibody to compete with and block another isolated antibody from binding to an allergen. When multiple unique isolated antibodies, e.g., candidate monoclonal antibodies, are tested for their ability to block one another from binding to the allergen, the test results reveal identifiable groups of antibodies, within which groups each antibody blocks the other group members from binding the allergen. An insight of the invention is that those groups of antibodies can be classified as distinct bins, dubbed epitope bins, within which member antibodies recognize overlapping epitopes on the allergen.

Additionally, methods of the invention include epitope binning, which groups antibodies that block one another from binding an allergen. Those methods comprise steps in which an antibody is bound to a surface and is exposed to a binding antigen. Antibodies in solution that also bind the same epitope of the antigen as the surface-bound antibody will be blocked from binding the antigen. Those that compete for binding at the epitope are part of the same bin. Binning allows an understanding of the number of epitopes of an antigen that are recognized by a given class of antibodies as well as the epitope specificity distribution. Subsequent to epitope binning, bins can be mapped to epitopes using, for example, linear peptide arrays. In mapping, certain antibodies from each bin are tested, alone and in combination, to determine whether the epitope to which the antibodies bind comprises, at least in part, a contiguous subsequence of amino acids, and if so, which subsequence(s). In combination, binning and mapping reveal which antibodies bind known immunodominant epitopes.

Once epitope bins are revealed for an allergen, the bins may be compared with one another, for example via an inhibition assay. An inhibition assay, such as a basophil activation test (BAT) or mast cell activation test (MAT), may serve as an in vitro surrogate for an allergic reaction upon exposure to the allergen of interest. Bins with antibodies that show, on average, higher abilities to inhibit cellular activation (or other desirable properties such as low autoreactivity) are selected. Proteins or other compounds that fall within a selected bin may be assessed for therapeutic efficacy to the exclusion of compounds that fall within unselected bins.

After one or more bins are selected, the ability of a candidate monoclonal antibodies in a particular bin to block a patient IgE antibody from binding to an allergen may be assessed to discriminate between candidates. It may be theorized that a candidate monoclonal antibody could be used therapeutically, and administered to a patient. If the therapeutic antibody blocks IgE from binding to allergen in the patient serum, the therapeutic antibody could block or inhibit the allergic response and potentially prevent anaphylaxis. The ability to block IgE may be measured by an in vitro competitive binding assay such as a blocking ELISA with a colorimetric result. An object of such a test is to identify candidate therapeutic antibodies that prevent colorimetric development resulting from antigen-IgE binding in the blocking ELISA.

When multiple unique isolated antibodies, e.g., candidate monoclonal antibodies, are tested from selected bins, each alone and in various combinations, for their ability to block antigen-IgE binding, a remarkable result obtained. Combinations of the candidate antibodies from selected bins may far outperform any single candidate antibody or combinations of antibodies from unselected bins.

### BRIEF DESCRIPTION OF THE DRAWINGS

FIG. 1 illustrates an IgE Blocking ELISA.

FIG. 2 diagrams an ELISA method to measure protein blocking of allergen-IgE binding.

FIG. 3 is a first part of results from a binning assay using anti-Ara h 2 antibodies.

FIG. 4 gives more of the results from the binning assay.

FIG. 5 shows the use of linear peptide microarrays for epitope mapping.

FIG. 6A shows an Ara h 2 model.

FIG. 6B shows the Ara h 2 model rotated 180 degrees.

FIG. 7 gives results showing compositions blocking recombinant Ara h 2 from binding IgE.

FIG. 8 illustrates one approach to selecting an antibody from within a desired bin.

FIG. 9 shows epitope binning for peanut Ara h 6 antibodies.

FIG. 10 shows the results of mast cell activation tests (MATs) against the Fel d 1 cat allergen.

### DETAILED DESCRIPTION

Allergy is an adaptive immune response to non-infectious substances referred to as allergens. Common allergens include food, mites, pollen, pet dander, mold, medicines, insect venom, and latex. Some allergic reactions such as contact dermatitis are thought not to involve IgE. However, many allergic responses and conditions such as anaphylaxis, allergic rhinitis (hay fever), food allergies, and allergic asthma, involve IgE and T helper 2 (TH2) cells that recognize antigens of allergens. In IgE-mediated responses, exposure to the allergenic substance may induce IgE production and sensitize the subject. The subject will then have circulating IgE immunoglobins specific to the allergen. Once threshold levels of IgE are met, the subject is susceptible to allergic reactions to the allergen in the future. If the subject later encounters the same allergen, they experience an allergic reaction.

Allergen exposure produces an acute reaction, which is known as an early-phase reaction or a type I immediate hypersensitivity reaction. An IgE-mediated type I immediate hypersensitivity reaction can occur within minutes of allergen exposure. In those cases, IgE bound to FcεRI on mast cells and basophils is crosslinked when multiple IgE/Fc complexes bind to an allergen. That cross-linking causes the mast cells and basophiles to degranulate, releasing mediators such as histamine and cytokines. Those mediators promote vasodilation, vascular permeability, and responses such as bronchoconstriction. Some of those mediators also promote the recruitment or activation of leukocytes, contributing to the development of subsequent “late-phase” reactions, with continuing inflammation and breathing trouble, over subsequent hours to days. The allergic reaction may involve natural feedback loops. For example, when basophils and mast cells degranulate in response to contact with IgE, they release IL-4, which may promote class-switching of B cells, potentially leading to increased production of IgE.

One approach to treating allergies clinically has involved immunotherapy, often in the form of oral immunotherapy in the case of food allergy. The goal of immunotherapy is to downregulate the immune response to antigen exposure. Some approaches to immunotherapy involve the slow introduction of antigen (e.g., of an allergen), often starting at low doses that increase over time. It may be found that such allergen exposure increases the subject's production of immunoglobin IgG4 specific to the allergen. It may be theorized that the IgG4 then competes with IgE to reach the target protein. That is, when the allergen is encountered, the IgG4 binds to an available epitope of the allergen, possibly blocking IgE from binding. Because IgE cannot bind allergen, even if IgE is bound to the Fc receptors on mast cells and basophils, nothing is available to bind the paratopes of the IgE molecules and cross-link multiple IgE-Fc receptor complexes. Because the IgG4 prevents the IgE/Fc complexes from being cross-linked, the IgG4 prevents mast cells and basophils from degranulating, and thus inhibits anaphylaxis. This suggests a protein product such as an antibody like an IgG4, or a fragment thereof, to be used as part of a therapeutic composition for the treatment of allergy.

Targets for allergy therapy may be recognized by understanding the cell and molecular mechanisms of allergy and of immunotherapy. For example, in some immunotherapies, an allergen is carried by antigen presenting cells to lymph nodes, which activates T cells and leads to the production of Tregs. Tregs suppress Th2 and stimulate growth of Th1 cells. Interaction between Tregs & B cells leads to the release of cytokines IL 10 and TGF-beta (from B cells and Tregs, respectively). As a result, B-cells are stimulated to produce IgG4. IgG4 binds to an allergen, thereby blocking it from binding to IgE on the surface of mast cells.

Therein lies an important insight, that IgG4 or anything else that blocks allergen from binding to IgE may prevent mast cells and basophils from degranulating, and thus inhibit anaphylaxis. Additionally, results presented herein show that allergens may be characterized by multiple epitopes and that compositions that include multiple monoclonal antibodies (or fragments thereof) that belong to two different epitope bins on an allergen far outperform the blocking of any single epitope. A significant insight from results of the invention is that two antibodies in combination may block IgE binding to an allergen substantially as well as combinations comprising antibodies that bind to three or more epitopes of the allergen.

In order to assure the most therapeutically effective multi-epitope-bin compositions, the methods and compositions of the invention rely on an early identification and selection of a single epitope bin or subset of epitope bins for an allergen from which antibodies are assessed. Accordingly, as certain epitope bins are selected, other epitope bins remain unselected. In this way, antibodies that are most effective at blocking IgE-allergen binding are discovered, without the need to screen and assay candidates from every bin. Moreover, by using only antibodies/fragments from a subset of epitope bins, high therapeutic efficacy may be achieved, while concurrently reducing manufacturing and development costs as well as the risk for unwanted side effects, such as cross- or auto-reactivity.

Preferred compositions of the invention include two or more monoclonal IgG4 antibodies, or allergen-binding fragments thereof. Embodiments of the invention seek to exploit a mechanism more effective than simple blocking, i.e., using multiple blocking antibodies assigned to a selected epitope bin or subset of epitope bins, as described and explained herein.

Methods of the invention may include, generally, (i) identifying epitope bins for a particular allergen; (ii) assessing the relative abilities of mAbs (or fragments thereof) in various bins (e.g., using an inhibition assay) to block IgE-allergen binding; selecting the epitope bins with the mAbs that show the greatest ability to block IgE-allergen binding; and screening only mAbs in the selected bins for use in a therapeutic.

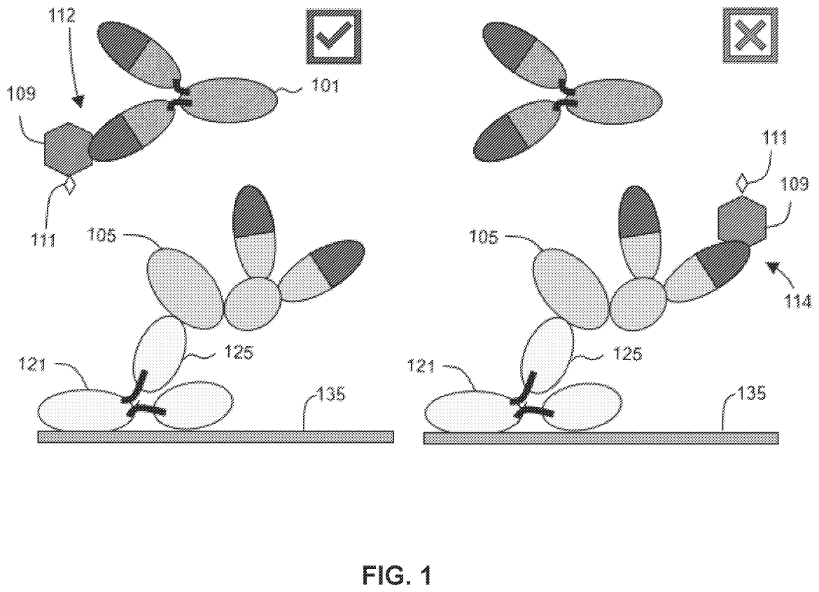

FIG. 1 illustrates an IgE Blocking ELISA designed to test competition of a therapeutic mAb-IgG4 101 with plasma IgE 105 for binding to an allergen 109. The test is whether binding 112 of the therapeutic antibody 101 to the allergen 109 successfully blocks binding 114 of IgE 105 to the allergen 109. By repeating the same blocking ELISA for multiple therapeutic candidates, blocking antibodies can be ranked.

Any suitable assay may be used in accordance with the invention to evaluate the ability of a mAb(s) (or fragment thereof) or a putative therapeutic to block binding of an allergen to IgE. In the illustrated approach, an allergen 109 with a His tag 111 is used. For the illustrated approach, IgE 105 is immobilized to a substrate 135 such as a wall or surface of a plate or well. Here, the IgE 105 is immobilized via the FceRI 125 of IgGI-Fc 121 fusion. As can be seen, if binding 112 of the therapeutic antibody 101 to the allergen 109 does not block binding of an allergen to IgE, then the His tag 111 is bound to the substrate 135 through the allergen 109, IgE 105, and FceRI 125-IgG1-Fc 121. Anti-His antibody-tagged enzyme & colorimetric substrate reveals allergen-IgE binding and, by implication, lack of enzyme activity shows bocking of allergen-IgE binding.

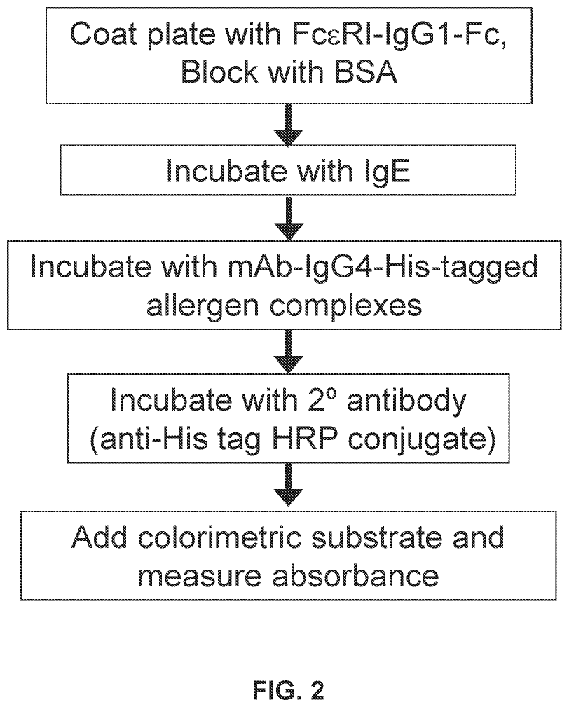

FIG. 2 diagrams steps of an ELISA protocol to measure blocking of allergen binding to IgE by one or any combination of therapeutic proteins. The protocol includes coating a plate with FceRI-IgG1-Fc and blocking with BSA. The coated plate is incubated with sample containing IgE, such as plasma or sera. The immobilized IgE is incubated with one or any combination of therapeutic proteins such as a therapeutic mAb-IgG4 complexed with His-tagged allergen. The reaction mix is incubated with a secondary antibody such as an anti-His tag HRP conjugate. A colorimetric substrate (ABTS or TMB) is added and absorbance is measured.

Of interest are relationships among therapeutic proteins and whether any of those products have additive effects when combined. Testing among monoclonal IgG4 candidate antibodies, using any appropriate assay, including the disclosed ELISA assay, at this stage reveals blocking of IgE-antigen interaction.

Methods of the disclosure may include evaluating obtained antibodies (or fragments thereof) for their ability to block one another from binding to an allergen and also for their ability (each alone or in combination) to block allergen from binding IgE. In fact, an important part of the disclosure includes that competitive blocking assays reveal blocking among sets of monoclonal antibodies in a manner used for epitope binning and then competitive blocking assays are used for evidence of the ability of those antibodies to block allergen-IgE binding. Additionally, blocking assays are used for epitope discovery as pairwise blocking assays among multiple antibodies reveals reproducible epitope bins and an individual antibody can be assigned to its epitope bin from those data.

Any suitable assay may be used to evaluate the ability of an antibody to block binding of other molecular species. In certain aspects, multiple antibodies against an allergen of interest are obtained and may be subject to epitope binning and/or used to define epitope bins.

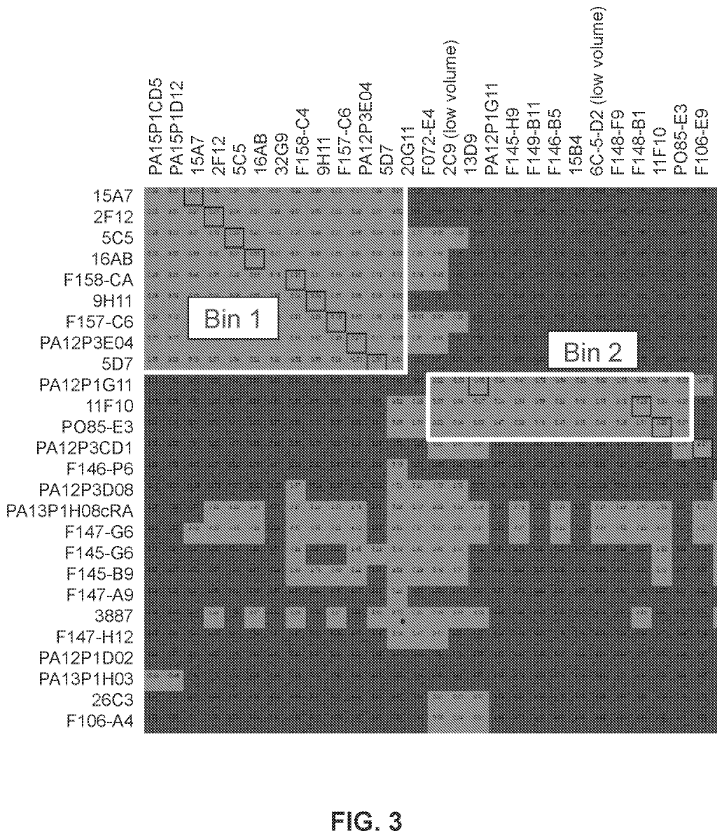

FIG. 3 provides epitope binning results for a set of anti-Ara h 2-antibodies. The column labels are PA15P1CD5; PA15P1D12; 15A7; 2F12; 5C5; 16AB; 32G9; F158-C4; 9H11; F157-C6; PA12P3E04; 5D7; 20G11; F072-E4; 2C9 (low volume); 13D9; PA12P1G11; F145-H9; F149-B11; F146-B5; 15B4; 6C-5-D2 (low volume); F148-F9; F148-B1; 11F10; PO85-E3; and F106-E9. The row labels are 15A7; 2F12; 5C5; 16AB; F158-CA; 9H11; F157-C6; PA12P3E04; 5D7; PA12P1G11; 11F10; PO85-E3; PA12P3CD1; F146-P6; PA12P3D08; PA13P1H08cRA; F147-G6; F145-G6; F145-B9; F147-A9; 3887; F147-H12; PA12P1D02; PA13P1H03; 26C3; and F106-A4.

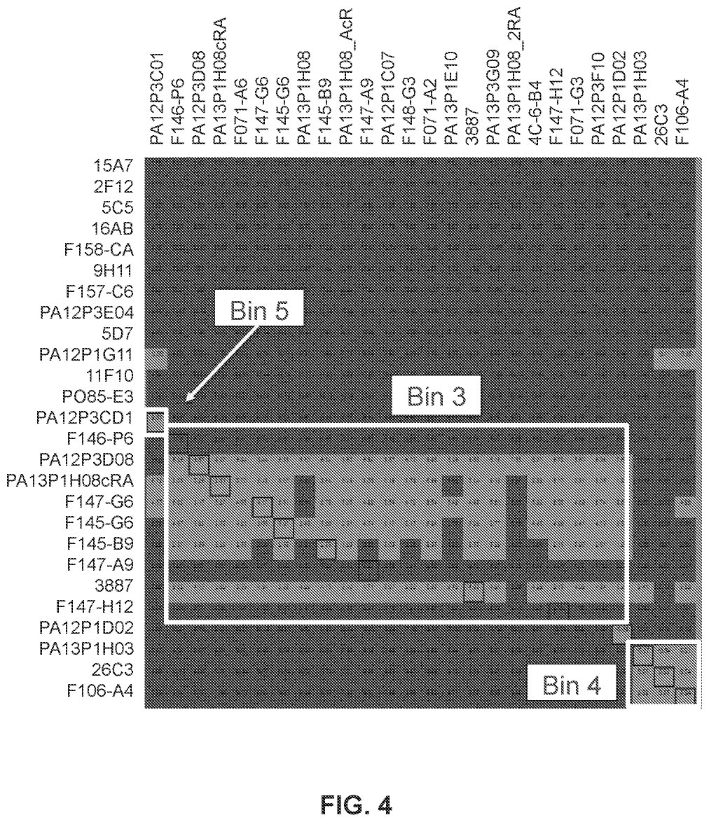

FIG. 4 provides additional anti-Ara h 2-antibody binning results. The column labels are PA12P3C01; F146-P6; PA12P3D08; PA13P1H08cRA; F071-A6; F147-G6; F145-G6; PA13P1H08; F145-B9; PA13P1H08_AcR; F147-A9; PA12PIC07; F148-G3; F071-A2; PA13P1E10; 3887; PA13P3G09; PA13P1H08_2RA; 4C-6-B4; F147-H12; F071-G3; PA12P3F10; PA12P1D02; PA13P1H03; 26C3; and F106-A4. The row labels are 15A7; 2F12; 5C5; 16AB; F158-CA; 9H11; F157-C6; PA12P3E04; 5D7; PA12P1G11; 11F10; PO85-E3; PA12P3CD1; F146-P6; PA12P3D08; PA13P1H08cRA; F147-G6; F145-G6; F145-B9; F147-A9; 3887; F147-H12; PA12P1D02; PA13P1H03; 26C3; and F106-A4.

The results shown in FIGS. 3-4 for the anti-Ara h 2-antibodies illustrate patterns indicative of distinct epitope bins. Antibodies in the abscissa in bin 1, for example, compete for the same epitopes as the antibodies in the ordinate of bin 1.

The results in FIGS. 3 and 4 show bins of antibodies that compete.

Thus, a bin is a set of antibodies in which each member tends to block the binding of other members of the bin. Note that certain things need not be known about the antibodies nor the antigen to identify the bins. The results shown in FIG. 3 and FIG. 4 were obtained using monoclonal antibodies against Ara h 2. Notably, for any given antibody, it is not necessary at to identify its cognate epitope on an allergen (in this case Ara h 2), and it is not necessary to identify the sequence of any part of the antibody. In fact, using epitope binning to measure blocking among the antibodies is sufficient to identify the bins in the results presented here.

It may be surmised that each bin defines a group of antibodies that share a target epitope. Remembering that the bins appear as rectangles crossed by a diagonal of antibodies being tested against themselves, these data suggest that epitopes of the allergen may be identified by, or assigned to, one of the bins.

Here, the data illustrate peanut Ara h 2 epitope binning. The data show five major bins on Ara h 2. FIG. 3 shows bins 1 and 2. FIG. 4 shows bins 3, 4, and 5. It is important to note that an antibody can be assigned to a bin even if the antibody does not appear on both the rows and the columns of the results. For example, the antibody 32G9 was not tested against itself but is assigned to Bin 1 on the basis of its blocking strength against the other members of Bin 1.

In general, for both classical and premixed epitope binning on the Carterra LSA (Carterra Bio, Salt Lake City, UT), surfaces were first functionalized with antibodies according to the following steps. Antibodies were coupled to HC30M sensor chips using 25 mM MES pH 5.5 (Carterra), 0.05% Tween-20 (Carterra) running buffer by dispensing with the LSA multichannel printhead. Sensor chips were activated with 133 mM 1-Ethyl-3-(3-dimethylaminopropyl) carbodiimide hydrochloride (EDC) (Pierce Premium Grade), 33 mM N-hydroxysulfosuccinimide (sulfo-NHS) (Pierce Premium Grade), 33 mM MES pH 5.5 (Carterra) buffer for 7 minutes. Antibodies were coupled to the activated surface at 0.1, 1.0, and 10.0 ug/mL concentration in 10 mM sodium acetate pH 4.75 (Carterra), 0.05% Tween-20 (Carterra). Remaining reactive groups were inactivated with 1 M ethanolamine pH 8.5 (Carterra) for 7 min. HBS-TE+0.05% BSA was used as the running buffer and diluent for all proteins.

For classical binning, each binding cycle consisted of 100 nM of nAra h 2 (Indoor Biotechnologies) injected using the LSA single flow cell with 4 minutes of association time, followed by 30 ug/mL of antibody with 4 minutes of association time, and regeneration with 2×10 sec injections of 0.65% phosphoric acid (Carterra). For premixed binning, 50 ug/mL of antibody was mixed with nAra h 2 (Indoor Biotechnologies) at a 10-fold molar excess, then injected using the LSA single flow cell with 4 minutes of association time. Analysis of epitope binning was performed using Epitope (Carterra). Binding of antibody was normalized to signal from the buffer-only injections that follow antigen injections for classical binning. Binding of antibody-antigen complex was normalized to antigen-only injections for premixed binning. Antibody pairs were classified as blocking or sandwiching according to whether they meet binding signal thresholds above buffer injection background. Blocking or sandwiching classification of antibody pairs were used to generate heat maps where blocking pairs are sorted into epitope bins. Any suitable methods may be used to obtain antibodies. Antibodies may be obtained from commercial or research libraries or product. Antibodies may be obtained from subjects, e.g., patients that have been exposed to allergen. Methods of the invention may include taking a sample (e.g., blood, plasma, serum, lymph, mucous, saliva, or any other suitable sample) from desensitized individuals who produce IgG4 against allergen.

As mentioned, for any given antibody, it is not necessary at to identify its cognate epitope on Ara h 2 in order to identify the bins. In fact, identification of the bins provides a useful tool for simplifying experiments involving mapping new antibodies to their epitopes, i.e., for discovering the epitope of an antibody.

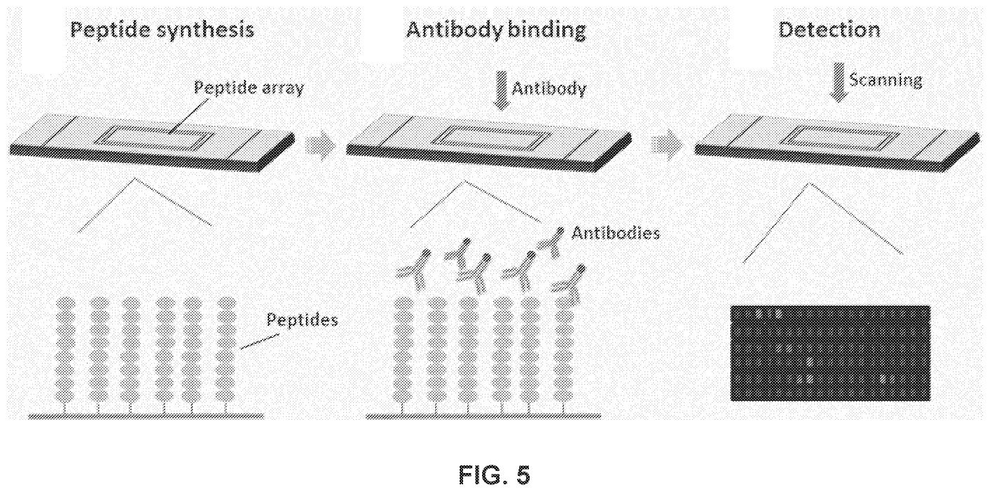

FIG. 5 shows the use of linear peptide microarrays as a tool to directly assign certain individual antibodies to their cognate epitopes. To assign antibodies to their epitopes, peptides are synthesized bound to a substrate in an array. While any suitable approach or variation of the method may be used, one approach is to then incubate the array with fluorescently-labeled antibodies. The antibodies are allowed to bind to the peptides that are linked to the substrate. Preferably, the peptides are synthesized with known sequences that represent short, epitope-length subsequences of the primary amino acid sequence of an allergen of interest. Preferably, the known sequences are synthesized at known spots on the array.

After the fluorescently-labeled antibodies are allowed to bind to the peptides on the array, the array is scanned to detect fluorescence from bound antibodies. For example, the array may be imaged using a fluorescence microscope. Fluorescent labels from the antibodies detected in the image may be assigned to locations on the array and correlated with the known peptide sequence of that location. By correlating a labeled antibody to the peptide to which it binds, where the sequence of that peptide is a short sub-sequence of the primary amino acid sequence of the allergen, that peptide sequence of the allergen can be putatively identified as the epitope of the antibody.

Linear peptide microarrays are used to identify epitopes of monoclonal antibodies against allergens of interest. A skilled artisan will be familiar with the concepts of linear and conformational epitopes as well as current thinking that there may be no bright line distinction. The linear peptide microarray suggests itself as a tool for mapping novel antibodies to linear epitopes.

The epitope bins presented herein provide a foundation for reducing the need for comprehensive epitope mapping of every antibody. Instead, one or more representative antibodies from each bin can be selected for epitope mapping. Methods for mapping antibodies to epitopes include obtaining a plurality of antibodies against an antigen, testing combinations of the plurality of antibodies for their strength of blocking one another, and identifying bins comprising sets of antibodies that block one another. At least a first epitope of the antigen is identified as the cognate epitope for a first antibody in a first bin. The epitope identification may be by any suitable method such as by a peptide assay such as a linear peptide microarray, hydrogen deuterium exchange, or crystallography. Each of the antibodies in the first bin is mapped to the first epitope by virtue of its membership in the first bin. In some embodiments, the first bin will include at least a second antibody that does not successfully bind to a linear peptide and thus is not assigned to a linear epitope in the peptide assay, e.g., the linear peptide microarray, ELISA sandwich assay, or such. The first epitope is identified as the cognate epitope of the second antibody on the basis of the identification of the first bin. For the second antibody, the first epitope may be reported as a non-linear epitope, and/or optionally may be denoted as a discontinuous epitope or a conformation epitope. Reporting the first epitope as a non-linear cognate epitope of the second antibody may be taken to mean that a substantial number of amino acids (e.g., 30% or 60% or more) in the first epitope participate in binding the second antibody to the antigen.

Methods are used to identify epitopes for antibodies raised against food allergens. For example, results from anti-Ara h 2 antibody binning assays reveal 5 major Ara h 2 epitope bins. Results from peptide assays (e.g., linear peptide microarrays) reveal that 4 epitopes are linear and one epitope is nonlinear. Methods are performed with Ara h 6 and 1 of the 4 linear epitopes are cross-reactive between Ara h 2 and 6 (additional data below). Methods further reveal that the non-linear epitope has a cognate antibody that is cross-reactive between Ara h 2 and 6.

Mapping of epitopes to antibodies using a peptide assay such as a linear peptide microarray may be shown, supported, or confirmed using 3D modeling.

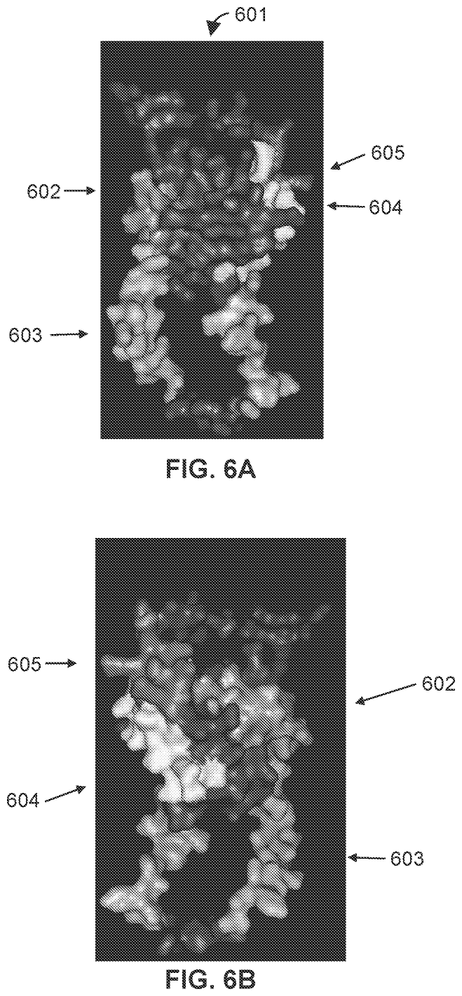

FIG. 6A shows an Ara h 2 model 601, i.e., a 3D model of Ara h 2 color-coded (or shaded) by epitope. The 3D model may be displayed from atomic coordinates such as found in a protein data bank file. Each atom may be assigned to an amino acid residue and amino acid residues may be grouped by bin, where a bin includes the cognate epitope(s) of a bin of antibodies that maximally block one another in a blocking assay. A group of amino acids that constitute a bin may be color-coded, or shaded, for display, to illustrate the epitope bins of the antigen. The Ara h 2 model 601 include epitope bin 602 (corresponding to Bin 2 on FIG. 3), epitope bin 603 (corresponding to Bin 3 on FIG. 4), epitope bin 604 (corresponding to Bin 4 on FIG. 4) and epitope bin 605 (corresponding to Bin 5 on FIG. 4).

FIG. 6B shows the Ara h 2 model 601 rotated 180 degrees about the vertical axis. Unmapped segments of Ara h 2 include R1-Q7, N20-R34, K121-R134, and E142-Y151. With reference to the antibody names used as labels on FIG. 3 and FIG. 4, Bin 1 on FIG. 3 is not shown on the Ara h 2 model but includes the antibodies 9H11 and F157-C6. Bin 2 on FIG. 3 includes the antibody F148-F9. Bin 3 on FIG. 4 includes the antibodies F071-A6 and F148-G3. Bin 4 on FIG. 4 includes the antibody F106-A4. Bin 5 on FIG. 4 includes the antibody PA12P3C01.

In some embodiments, methods of the invention include obtaining sample from a person who is currently allergic or has become desensitized to an allergy. Any suitable people or population may be sampled. For example, subjects who have undergone oral immunotherapy and become desensitized may represent a suitable population to obtain samples from. Some such subjects may have participated in oral immunotherapy as a cohort in a study sponsored by an institution, or through participation in a treatment program administered by an institution, and in such cases the institution may have a “bank” or “collection” of samples, e.g., blood or plasma or serum samples from those subjects. Such a collection of samples may be surveyed or assayed to identify factors that contribute desensitization. Methods may include analyzing a sample to identify a molecular species that inhibits an allergic response.

In some embodiments, RNA-seq is performed on B cells isolated from the peripheral blood of food allergic or desensitized individuals, and each cell's gene expression, splice variants, and heavy and light chain antibody sequences are characterized. Blood may be separated into plasma and cellular fractions; plasma stored and later used for allergen-specific immunoglobin concentration measurements, while the cellular fraction may be enriched for B cells prior to FACS. CD19+B cells of may be sorted exclusively based on immunoglobulin surface expression, but with an emphasis on IgE and/or IgG4 B cell capture. Isotype identity may be determined from scRNA-seq. B cell capture by such methods avoid stringent requirements on FACS gate purity or the need for complex gating schemes. Single cells may be sorted into wells or other fluid partition, e.g., droplets on a microfluidics platform, and processed using a modified version of the Smart-seq2 protocol. See Picelli, 2014, Full-length RNA-seq from single cells using Smart-seq2, Nat Protocol 9:171-181, incorporated by reference. Sequencing may be performed on an Illumina NextSeq 500 with 2×150 bp reads to an average depth of 1-2 million reads per cell. Sequencing reads may be aligned and assembled to produce a gene expression count table and/or to reconstruct antibody heavy and light chains, respectively. Using software such as STAR for alignment also facilitates the assessment of splicing within single cells. See Dobin, 2013, STAR: ultrafast universal RNA-seq aligner, Bioinformatics 29:15-21, incorporated by reference. Cells may be stringently filtered to remove those of low quality, putative basophils, and those lacking a single productive heavy and light chain. Isotype identity of each cell may be determined by its productive heavy chain assembly, which avoids misclassification of isotype based on FACS immunoglobulin surface staining. From such sequences, the sequences of antibodies such as IgG4 and/or IgE may be determined. Such sequences could be cloned and expressed recombinantly. See Dodev, 2014, A tool kit for rapid cloning and expression of recombinant antibodies, Scientific Reports 4:5885, incorporated by reference.

Methods of making and purifying antibodies are known in the art and were described by Harlow and Lane, 1988, Antibodies: A Laboratory Manual, CSHP, Incorporated by reference. Antibodies (e.g., IgG4 and/or IgE) may be produced recombinantly or generated using hybridoma technology, wherein isolated B lymphocytes in suspension are fused with myeloma cells from the same species to create monoclonal hybrid cell lines that are virtually immortal while still retaining their antibody-producing abilities. See Harlow and Lane, 1988, Antibodies: A Laboratory Manual, CSHP, incorporated by reference. Such hybridomas may be stored frozen and cultured as needed to produce the specific monoclonal antibody. Such monoclonal antibodies may be deployed therapeutically in methods of the invention. Those immunoglobins may exhibit single-epitope specificity and the hybridoma clone cultures provide an unchanging supply over many years. Hybridoma clones may be grown in cell culture for collection of antibodies from the supernatant or grown in the peritoneal cavity of a mouse for collection from ascitic fluid.

Having obtained a plurality of monoclonal antibodies against an antigen and mapped epitopes to the obtained antibodies according to methods of the invention, one or more of the plurality of monoclonal antibodies is selected to be included in a therapeutic composition for the treatment of allergy or the inhibition of anaphylaxis.

It is understood that anaphylaxis is a potentially life-threatening aspect of an allergic response to an allergen. Anaphylaxis may result when a sensitized patient ingests and allergen, and antigen of the allergen encounters IgE antibodies in the patient. When multiple antigen-specific IgE antibodies bind to an antigen and cross-link receptors on the patient's mast cells, the cross-linking may lead to degranulation of the mast cells, which degranulation forms a central part of the anaphylactic response.

Accordingly, the invention provides a method of inhibiting anaphylaxis. The method includes administering to a subject a composition comprising a first antibody, or fragment thereof, that binds to an allergen and a second antibody, or fragment thereof, that binds to the allergen, wherein the first antibody and the second antibody do not block one another from binding to the allergen, thereby blocking IgE antibodies of the subject from binding to the allergen.

The first antibody and second antibody may be monoclonal antibodies, e.g., monoclonal IgG4 antibodies. The method may be used to target Ara h 2 epitopes from two bins. The Ara h 2-specific mAbs are high affinity and, in multi-bin combination, block IgE binding to rAra h 2 in vitro. Method may include dosing at a dosage between about 100 mg and 300 mg by subcutaneous injection every few weeks (e.g., SCq4w) in participants with a medically confirmed diagnosis of IgE-mediated peanut allergy. Embodiments of the method use mAbs from multiple bins to inhibit peanut-mediated cellular degranulation in vivo. The composition is administered to block allergic patient sera IgE from binding to peanut protein with sub-nM IC50. Preferably the method inhibits activation of IgE sensitized basophil and/or mast cell exposed to peanut protein by >70% with sub-nM IC50. Methods may exhibit broad activity in all patients allergic to peanut protein. Certain embodiments include subcutaneous administration. Benefits of the disclosed antibodies used in combinations from 2 or more bins include predictable, minimal toxicities with no human tissue cross-reactivity.

Antibodies can be produced in animals, i.e., by immunization of an animal with an allergen. Once the sequence of the allergen is known, it can be cloned, e.g., into yeast or bacteria, and grown up in bulk to form a protein product that primarily includes the allergen for use in animal immunization to raise blocking antibodies. The protein product can be harvested from the growth vector and inoculated into animals (e.g., mice) to cause them to grow antibodies against the allergen. Those antibodies may be harvested and optionally sequenced and/or cloned via hybridoma technology for further expansion, e.g., followed by isolation for use in a therapeutic composition.

A composition of the invention is useful for the treatment of allergy when the composition includes components that prevent an antigen from binding to IgE in the patient. Any suitable assay may be used to evaluate the ability of a therapeutic to block binding of an allergen to IgE. For example, a plurality of monoclonal antibodies raised against an antigen may be tested via an IgE Blocking ELISA designed to test competition of a therapeutic antibody with plasma IgE for binding to an allergen. As discussed previously, an ELISA protocol may be used to measure blocking of one or any combination of therapeutic proteins with IgE. The protocol may include coating a plate with FceRI-IgG1-Fc and blocking with BSA. The coated plate is incubated with IgE. The immobilized IgE is incubated with one or any combination of therapeutic proteins such as a therapeutic mAb-IgG4 complexed with His-tagged allergen. The reaction mix is incubated with 2° antibody such as an anti-His tag HRP conjugate. A colorimetric substrate (ABTS or TMB) is added and absorbance is measured.

As the test protein is added, if it does not block antigen binding to IgE, then the His tag will capture an anti-His tag HRP conjugate, and that spot or well will generate an optical signal. In the described in vitro assay, a therapeutic protein that blocks IgE binding will decrease the optical signal. A decreasing optical signal with increasing protein concentration is an indication of an effective mechanism to block IgE binding by antigen. Because IgE binding by antigen is an in vivo condition for cross-linking of IgE on mast cells and anaphylaxis, compositions that decrease the optical signal in the described in vitro assay are indicated to be useful therapeutics for the treatment of allergy and anaphylaxis.

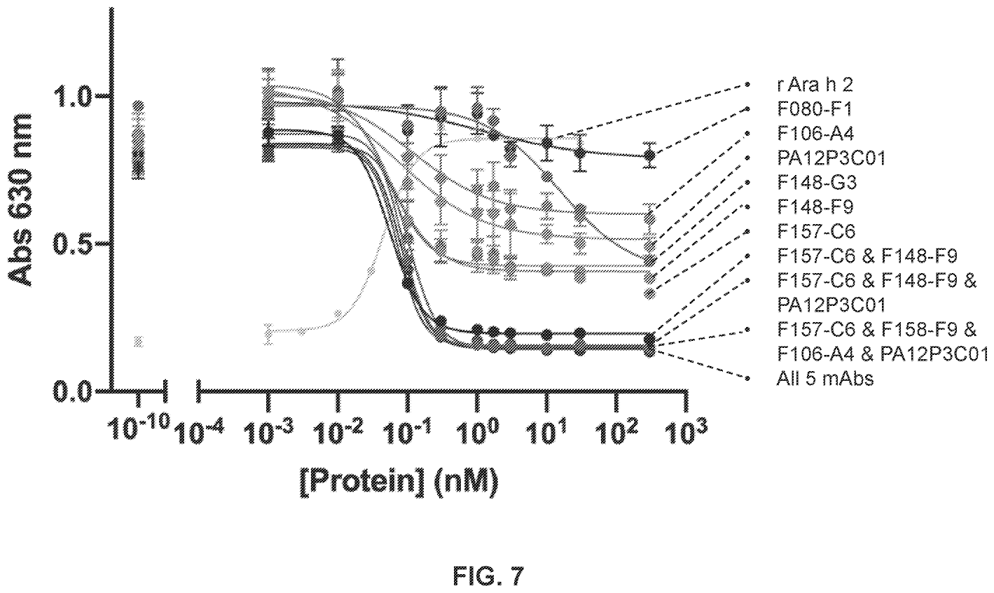

FIG. 7 shows results of an assay to measure the ability of a composition to block antigen from binding IgE. A recombinant Ara h 2 ELISA blocking assay was performed with a colorimetric indicator. A 630 nm optical signal was measured for eleven compositions that included monoclonal IgG4 antibodies selected based on epitope binning results presented in FIG. 3 and FIG. 4. Each composition tested, besides control antibody F080-F1, decreased optical signal at increasing concentrations of the protein antibody.

In the depicted results from the ELISA blocking assay, four of the traces show the greatest decrease in optical signal. Those four traces correspond to the following four combinations of monoclonal antibodies: (1) F157-C6 & F148-F9; (2) F157-C6 & F148-F9 & PA12P3C01; (3) F157-C6 & F158-F9 & F106-A4 & PA12P3C01; and (4) all 5 (F157-C6, F148-F9, F148-G3, F106-A4, PA12P3C01).

The presented results show an important insight of the invention. A combination of antibodies that includes members from any two epitope bins provides significantly better results than any single antibody. Moreover, once two bins are represented, it may be that including antibodies from one or more additional bins does not improve the results to as great an extent as provided by including antibodies from at least two bins. This is an important insight.

Once two epitopes are blocked by cognate therapeutic antibodies, even for an allergen with three, four, five or more identified epitopes, there may be significant diminishing returns in making compositions with cognate antibodies to greater than two of the epitopes. Results show that compositions are effective at blocking IgE from binding to allergen when the compositions include first and second antibodies that in combination block IgE binding to the allergen. The results further show that the combination of two blocks IgE substantially as well as combinations comprising antibodies that bind to three or more epitopes of the allergen. Combinations with three (or four, or five) follow essentially the same trace along FIG. 7 as the combination that includes two. Compositions of the invention treat allergy by including a first antibody, or fragment thereof, that binds to a first epitope on an allergen having more than two epitopes and a second antibody, or fragment thereof, that binds to a second epitope on the allergen, wherein addition of third or subsequent antibodies does not significantly reduce IgE binding as compared to a combination of the first and second antibodies. The traces for three antibodies, or four, or five, lie within <10% difference in absorbance at 630 nm. A quantitative result of measured IgE blocking in a benchtop, in vitro assays such as a blocking ELISA shows that using two antibodies that do not compete with one another gets to about 90% of the value possibly given by the assay, and adding a third or subsequent antibody does not add anywhere near 10% to that quantitative result. The third and subsequent antibodies add, at best, about 1% to the result in an in vitro quantitative assay for IgE blocking, which results are shown in FIG. 7.

FIG. 6A and FIG. 6B, viewed in light of FIG. 3 and FIG. 4 show that methods herein may be used to map therapeutic proteins such as monoclonal antibodies to their cognate epitopes and to identify a number of available epitopes of an allergen. The clearly defined bins of FIG. 3 and FIG. 4 were used to code the model in FIG. 6A, which shows that Ara h 2 (an important peanut allergen) has at least five distinct epitopes.

Another insight of the invention is that a composition that includes at least one of the monoclonal antibodies for each of the five bins (e.g., F-080-F1, F106-A4, PA12P3C01, F148-G3, and F148-F9) will bind to all of the epitopes on the allergen. Compositions composed this way may offer maximal IgE blocking, allowing no sites on the allergen to available to the patient's allergic immune response. However, as shown, binding all epitopes, versus a subset of epitopes, on an allergen may lead to diminishing returns—highlighting the importance of identifying promising bins to avoid screening mAbs from less effective bins.

FIG. 6A and FIG. 6B also shows the availability of therapeutic proteins that bind to multiple distinct epitopes. The results show that epitope mapping binning was performed to identify at least five epitope bins on Ara h 2. The mapping data, based on a blocking assay, reveal in that case that proteins of bin 3 bind a repetitive, linear motif on Ara h 2.

Thus, the invention provides compositions that include a therapeutic protein that binds a number of available epitopes on an allergen. The therapeutic protein can be used alone to bind and block multiple, potentially repeating, epitopes or may be used in combination with other therapeutic proteins.

Using the benefits and insights provided by those results, the invention provides a pharmaceutical composition for the prevention or treatment of allergy. The composition includes a first antibody, or fragment thereof, that binds to an allergen and a second antibody, or fragment thereof, that binds to the allergen, wherein the first antibody and the second antibody do not block one another from binding to the allergen. The statement that the first antibody and the second antibody do not block one another from binding to the allergen is a corollary to the provision that the first and second antibodies are selected from two different bins, as described above. The first antibody and second antibody are selected so that the first epitope and the second epitope are from different epitope bins, where each epitope bin is defined to include antibodies that block each other from binding. Accordingly, the composition embodies the insights presented in FIG. 7 that using antibodies from any two epitope bins does a markedly better job of blocking antigen from binding to IgE than does any single antibody alone.

Nevertheless, selecting the most effective bins from which to obtain multiple mAbs is more effective than selecting multiple mAbs across any or all bins.

Thus, methods and compositions of the disclosure include antibodies that have been mapped to two different epitope bins. The first antibody binds a first epitope of the allergen and the second antibody binds a second epitope of the allergen. Understood visually with reference to FIG. 6A, the first antibody and the second antibody would bind to the model Ara h 2 601 at different locations on the model in 3D space. This is a consequence of the insight that the first epitope and the second epitope are formed by different portions of the allergen. By performing the epitope mapping as described, the first epitope and the second epitope may be selected for minimal cross-reactivity with the second antibody and the first antibody. In peanut embodiments, the allergen targeted by the methods may be Ara h 2 and the first epitope and the second epitope may be different and each bind to an epitope on Ara h 2 that includes one of the following sets of residues of Ara h 2: 1-9; 10-18; 21-30; 31-39; 40-54; 55-60; 109-117; 121-130; and 137-146. In other embodiments, the allergen is from milk, egg, tree nut, fish, shellfish, soy, legume, seed, wheat, cat, birch, latex, pollen, or fungus.

Once a plurality of antibodies are assigned to bins, methods may be used to select bins with the most therapeutically effective mAbs.

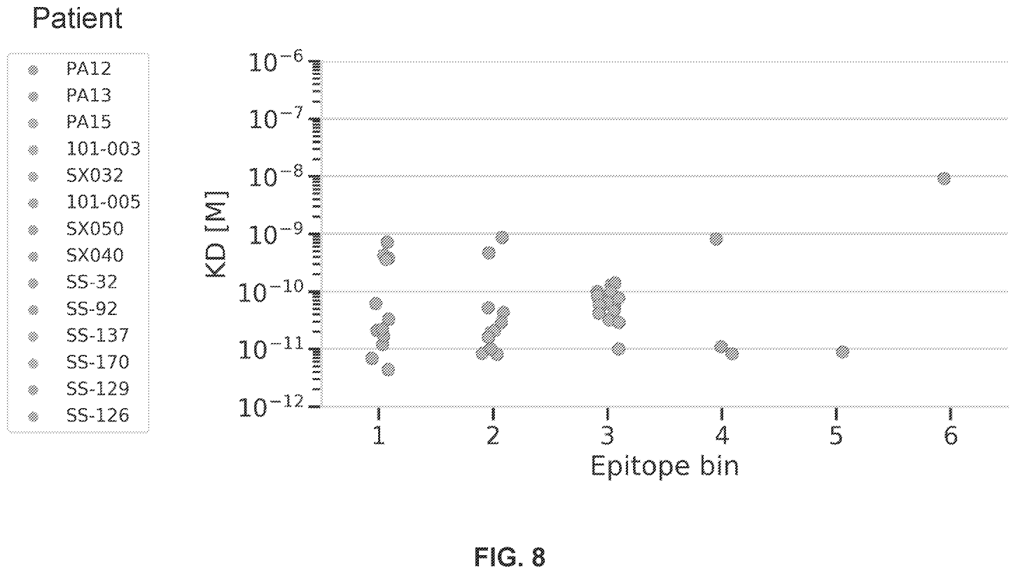

FIG. 8 illustrates one the affinities of antibodies to Ara h 2 across various bins. In FIG. 8, a plurality of antibodies have been assigned to epitope bins and, for each antibody, a dissociation constant (KD) has been measured for antibody-antigen binding. Note that these KD assays may be performed with antibodies obtained from patients. An important features is to measure KD for antibodies of interest. Guidance suggests that a KD of 10\hat{ }-9 M=1 nM is a useful therapeutic cutoff and antibodies should plot beneath that line. Compositions of the disclosure include high-affinity antibodies that plot beneath the KD=10\hat{ }-10 M=100 pM. Thus compositions of the invention preferably include high affinity IgG4 monoclonal antibodies from at least 2 different bins.

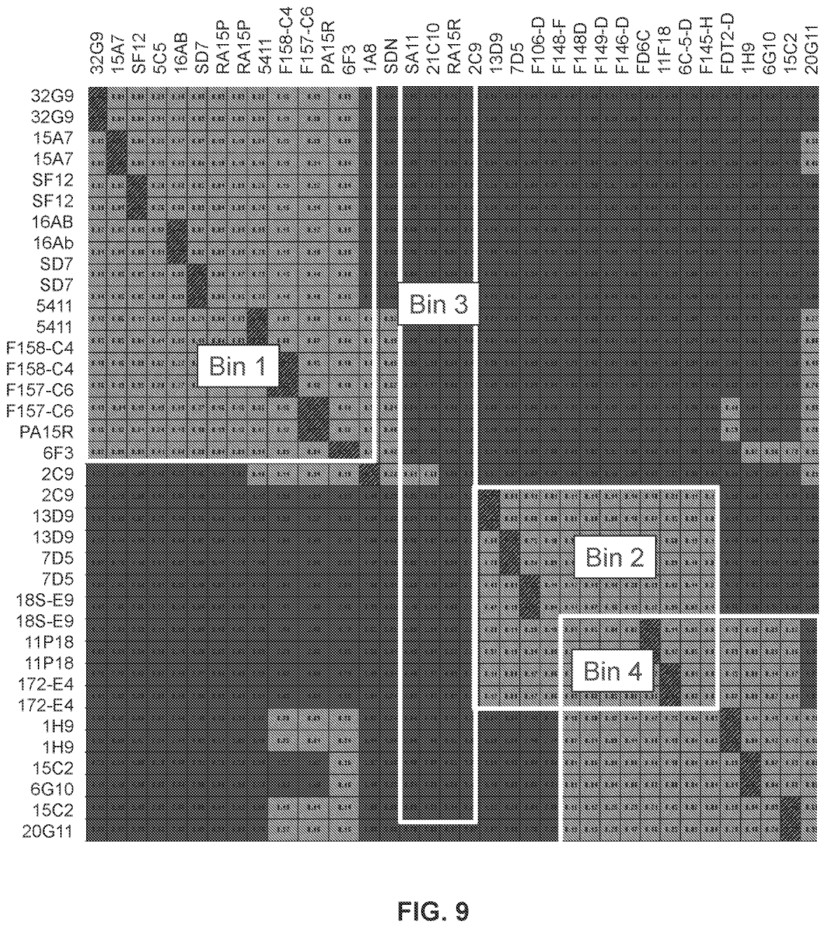

FIG. 9 shows epitope binning for antibodies specific to peanut Ara h 6. In FIG. 9, the column labels include 32G9; 15A7; SF12; 5C5; 16AB; SD7; RA15P; RA15P; 5411; F158-C4; F157-C6; PA15R; 6F3; 1A8; SDN; SA11; 21C10; RA15R; 2C9; 13D9; 7D5; F106-D; F148-F; F148D; F149-D; F146-D; FD6C; 11F18; 6C-5-D; F145-H; FDT2-D; 1H9; 6G10; 15C2; and 20G11. The row labels include 32G9; 32G9; 15A7; 15A7; SF12; SF12; 16AB; 16Ab; SD7; SD7; 5411; 5411; F158-C4; F158-C4; F157-C6; F157-C6; PA15R; 6F3; 2C9; 2C9; 13D9; 13D9; 7D5; 7D5; 18S-E9; 18S-E9; 11P18; 11P18; 172-E4; 172-E4; 1H9; 1H9; 15C2; 6G10; 15C2; and 20G11. The unique entries are 5411; 11F18; 11P18; 13D9; 15A7; 15C2; 16AB; 172-E4; 18S-E9; 1A8; 1H9; 20G11; 21C10; 2C9; 32G9; 5C5; 6C-5-D; 6F3; 6G10; 7D5; F106-D; F145-H; F146-D; F148D; F148-F; F149-D; F157-C6; F158-C4; FD6C; FDT2-D; PA15R; RA15P; RA15R; SA11; SD7; SDN; and SF12. Like the other figures, the image in FIG. 9 is a heat map, using shades of gray to represent relative blocking strength, e.g., Bin 1 (or for that matter, the other bins) are a different shade of gray that the surrounding areas of the heat map.

The discussion above for Ara h 2 applies here for interpreting labels, identifying bins, and assigning antibodies to epitope bins. The results from Ara h 6 binning show ˜3-4 bins (here labeled as 4 bins). Notably, Bins 1 and 2 are the “same” bins as for Ara h 2 i.e., the mAbs are cross-reactive to both allergens. Bins 3 and 4 are Ara h 6-specific.

In another important point, FIG. 9 shows that Ara h 6 was discovered to include at least 4 available epitope bins. The invention provides compositions that include multiple (e.g., at least 4) therapeutic proteins (e.g., monoclonal IgG4 antibodies) that bind to four epitopes on Ara h 6.

According to methods of the disclosure, monoclonal antibodies discovered from human allergic donors are high affinity (e.g., sub-nanomolar KD). 78% of those that bind Ara h 2 have KD of ≤100 pM (of which 9 have a KD of ≤10 pM). Affinities for Ara h 6 are similarly as low as the ˜10 pM—single digit nM range. High-affinity mAbs originate from numerous subjects. Individuals produce mAbs against multiple epitopes. Ara h 2 has at least 5 epitopes, 2 of which cross-react with Ara h 6. Ara h 6 has at least about 4 epitopes, 2 of which cross-react with Ara h 2. Some identified Ara h 2 epitopes (see, e.g., FIG. 6A) correspond to those found in literature. An insight of the disclosure is that representing any two bins in a composition, as shown in FIG. 7, provides better results than using antibodies targeted to a single epitope.

In preferred aspects, an inhibition assay, such as a mast cell activation test (MAT) or a basophil activation test (BAT), is used to assess the therapeutic potential and/or efficacy of mAbs in different epitope bins to identify bins of interest. A BAT is a flow cytometric assay which detects the ability of IgE to activate basophils stimulated by exposure to an allergen. A BAT generally measures the expression of activation markers (usually CD63 or CD203c) on basophil cell membranes (or membranes of other effector cells, such as mast cells) following cross-linking of IgE antibodies due to introduction of an allergen of interest. When in a resting state, an activation marker is expressed at low levels and is mainly found inside cell granules. Upon exposure to an allergen, the activation marker it is rapidly upregulates and the granules fuse with the basophil cell membranes, exposing the marker on the cell surface where it is detected by labelled antibodies with subsequent flow cytometry. The MAT is a similar assay in which mast cells are used instead of basophils.

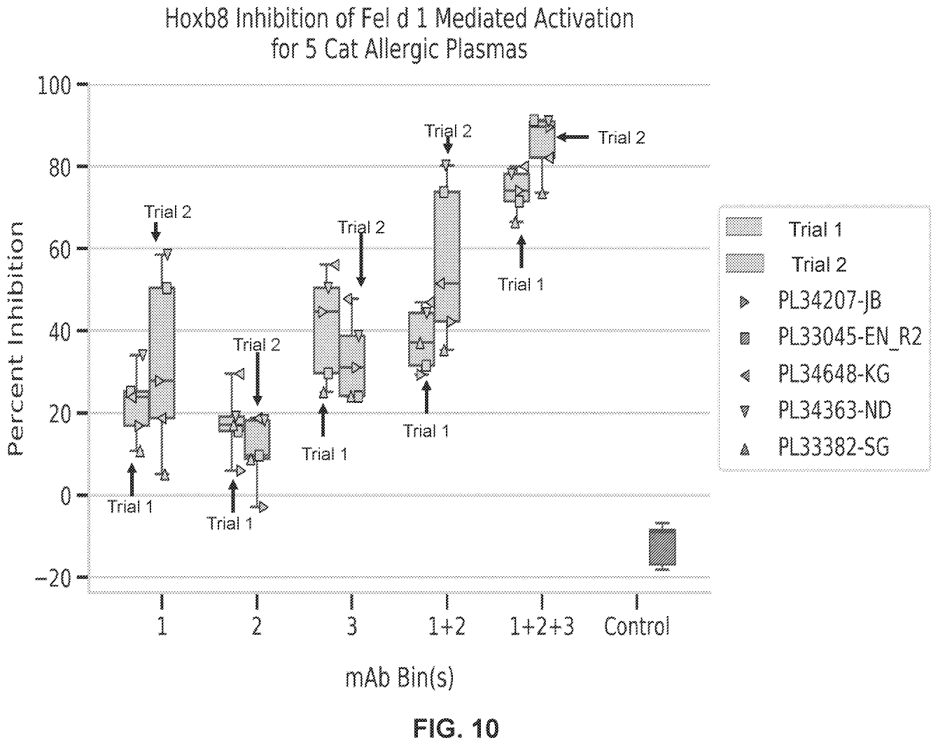

FIG. 10 shows the results of a MAT using mAbs from various Fel d 1 epitope bins. As shown, like Ara h 2, multiple epitope bins were identified and mapped for Fel d 1—in this case, Fel d 1 has three distinct allergen bins. In this MAT Hoxb8 cell were used, which are an immortalized progenitor line from the bone marrow of mice that are transgenic for the human high-affinity IgE receptor (FcεRIα) and can be reproducibly differentiated into mature Hoxb8 mast cells.

In the MAT of FIG. 10, the Hoxb8 cells were contacted with: (i) a mAb from epitope bin 1; (ii) a mAb from epitope bin 2; (iii) a mAb from epitope bin 3; (iv) mAbs from epitope bins 1 and 2; or (v) mAbs from epitope bins 1, 2 and 3. A BAT was repeated across these bins/combination of bins with different mAbs from each bin (i.e., “Trial 1” and “Trial 2”). For “Trial 1” mAbs were incidentally discovered from subjects that were unaware of a cat allergy, yet nonetheless has mAbs that bound to Fel d 1. It should be noted that subjects specifically recruited for a cat allergy are expected to yield higher affinity mAbs. The mAbs from bins 1 and 2 for “Trial 2” were obtained from a therapeutic currently undergoing clinical trials. This therapeutic includes mAbs from bins 1 and 2, but not bin 3. In “Trial 2”, a mAb from epitope bin 3 was added for the test of epitope bins “1+2+3”. For each trial the Hoxb8 cells were sensitized to five different allergic patient plasma samples (PL34207-JB, PL33045-EN_R2, PL34648-KG, PL34363-ND, and PL33382-SG) that were stimulated with Fel d 1.

As shown, in FIG. 10, individual mAbs from epitope bins 1 and 3 showed the highest levels of inhibition in the MAT (with bins one and three being the highest) and thus offer the best potential therapeutic efficacy. As described hereinabove, therapeutics targeting a combination of allergen epitopes often provide greater IgE-allergen blocking than those targeting a single epitope. This generally holds true in FIG. 10, in which MATs of epitope bins 1+2 and 1+2+3 tended to show greater inhibition than the single epitope bin MATs.

However, that is not always the case, and reveals the critical insight of the present invention—the mAbs of certain epitope bins provide better efficacy than the mAbs from other bins. As stated, mAbs from epitope bin 3 showed higher inhibition relative to the mAbs from epitope bins 1 and 2. For certain plasmas and mAbs, epitope bin 3 mAbs provided comparable or even higher levels of inhibition relative to combination treatments using mAbs from epitope bins 1+2. Moreover, for the epitope bin 1+2 mAbs used in the therapeutic undergoing clinical trials (“Trial 2”), as described above, for 3/5 patient plasmas, the therapeutic the mAbs provided less than 55% inhibition. By adding mAbs from epitope bin three to the epitope bin 1+2 mAbs, inhibition levels increased dramatically across all activated patient plasmas.

Thus, by not identifying the most efficacious epitope bins, it appears that considerable development cost has been sunk on a Fel d 1 therapeutic (the “Trial 2” bin 1+2 mAbs) that is only moderately effective. In contrast, using methods of the invention, it is clear that mAbs from epitope bins 1 and 3 are far more efficacious relative to epitope bin 2. Thus, the most effective combination is a therapeutic using the mAbs (or fragments thereof) from epitope bins 1 and 3.

Having obtained a plurality of monoclonal antibodies against an antigen, optionally mapped to epitopes, with the most efficacious bins identified, one or more of the plurality of monoclonal antibodies from the selected bins may be selected to be included in a therapeutic composition for the treatment of allergy or the inhibition of anaphylaxis.

Thus, the invention provides compositions that include a therapeutic protein that binds epitopes corresponding to the most immunodominant epitope bins of an allergen.

Using the benefits and insights provided by those results, the invention provides a pharmaceutical composition for the prevention or treatment of allergy. The composition includes a first antibody, or fragment thereof, that binds to an allergen and a second antibody, or fragment thereof, that binds to the allergen. The first antibody and second antibody are selected as the most efficacious subset of bins (e.g., from an allergen with three or more epitope bins) so that the first epitope and the second epitope are from different epitope bins, where each epitope bin is defined to include antibodies that block each other from binding. Accordingly, the composition embodies the insights presented in FIG. 10 that using antibodies from the most efficacious epitope bins does a markedly better job of blocking antigen from binding to IgE than does any single antibody alone or combinations from less efficacious bins.

As supported by the results shown in FIG. 10, the present invention provides therapeutic compositions that target Fel d 1 based on mAbs (or fragments thereof) from epitope bins 1 and/or 3 to the exclusion of those from epitope bin 2. The present invention also provides therapeutic compositions whereby a more inhibitory bin 2 antibody is included with a bin 1 and/or bin 3 antibody.

The molecular species (e.g., antibody) may then be prepared for therapeutic delivery. Antibody therapeutics may be given systemically by intravenous (IV) injection or by intramuscular (IM) and subcutaneous (SC) injection modes. Preferably the therapeutic is formulated at a suitably high stable concentration with parameters such as viscosity optimized for a delivery route. For example, viscosity maybe tuned to match a particular syringe or autoinjector. The formulation may comprise an aqueous solution or suspension with suitable buffers and optionally any other excipients to mitigate undesirable protein instability. Example excipients include fillers, extenders, diluents, solvents, preservatives, absorption enhancers and sustained release matrices. Buffers and excipients that are FDA approved for formulation of antibodies are generally known by those of skill in the art.

Once the therapeutic composition has been formulated, it may be provided for delivery to a patient who is potentially susceptible to an allergic reaction. For example, the composition may be packaged, e.g., in a bottle, vial, syringe, autoinjector, reservoir for an autoinjector or other device, or IV bag. The composition may be stored, e.g., in a cooler or freezer, or carried to a clinical setting for delivery. The composition may be packaged, e.g., in dry ice, and shipped to a hospital or other clinical setting for administration to a subject by a clinical professional.

In compositions of the invention, the antibodies, or fragments thereof, may be present in any suitable form and with any suitable modification. For example, the composition may include only the fragment of either antibody that binds antigen. The composition may include only a Fab region of either or both antibody, or even a subset of the Fab region. The Fab region may be present in the full antibody, with hinge and Fc region (partially or in the entirety). Any part of the molecular structure maybe modified, e.g., with substitutions or linkage to additional molecular structures.

In preferred embodiments, either of the first antibody and second antibody are monoclonal antibodies. The first antibody and/or second antibody may be IgG antibodies, e.g., IgG4. In some embodiments, the first antibody and second antibody are in an aqueous suspension comprising the monoclonal IgG4 in a pharmaceutically acceptable excipient or buffer. In various embodiments, the allergen specificity of the antibody(ies) is from one selected from the list consisting of: peanut, milk, egg, tree nut, fish, shellfish, soy, legume, seed, wheat, cat, birch, latex, pollen, and fungus.

Accordingly, the invention further provides methods for inhibiting anaphylaxis. An exemplary method includes administering to a subject a composition comprising a first antibody, or fragment thereof, that binds to an allergen and a second antibody, or fragment thereof, that binds to the allergen, wherein the first antibody and the second antibody are from the two most efficacious epitope bins identified for an allergen, thereby blocking IgE antibodies of the subject from binding to the allergen.

The first antibody and second antibody may be monoclonal antibodies, e.g., monoclonal IgG4 antibodies. The method may be used to target epitopes from a subset of potential epitope bins with an identified potential efficacy. Methods may include dosing at a dosage between about 100 mg and 600 mg by subcutaneous injection every few weeks in participants with a medically confirmed diagnosis of an IgE-mediated allergy, in particular an Fel d 1 allergy. Embodiments of the method use mAbs from multiple bins to inhibit Fel d 1-mediated cellular degranulation in vivo. The composition is administered to block allergic patient sera IgE from binding to Fel d 1 protein. Preferably the method inhibits activation of IgE sensitized basophil and/or mast cell exposed to Fel d 1 protein by >70%. Methods may exhibit broad activity in all patients allergic to Fel d 1. Certain embodiments include subcutaneous administration. Benefits of the disclosed antibodies used in combinations from 2 or more bins include predictable, minimal toxicities with no human tissue cross-reactivity.

Preferred embodiments use two or more monoclonal IgG4 antibodies, or allergen-binding fragments thereof. Embodiments of the invention seek to exploit a mechanism more effective than simple blocking, i.e., using multiple blocking antibodies assigned to different epitope bins, as described an explained herein.

Throughout the present description it is understood that methods of the inventions may be used to respond to, study, or treat allergies to any of the following allergens; Ambrosia artemisiifolia (short ragweed) antigen E (Amb a 1); Ambrosia artemisiifolia (short ragweed) antigen K (Amb a 2); Ambrosia artemisiifolia (short ragweed) Ra3 antigen (Amb a 3); Ambrosia artemisiifolia (short ragweed) Ra5 antigen (Amb a 5); Ambrosia artemisiifolia (short ragweed) Ra6 antigen (Amb a 6); Ambrosia artemisiifolia (short ragweed) Ra7 antigen (Amb a 7); Ambrosia trifida (giant ragweed) Ra5G antigen (Amb t 5); Artemisia vulgaris (mugwort) antigen (Art v 1); Artemisia vulgaris (mugwort) antigen (Art v 2); Helianthus annuus (sunflower) antigen (Hel a 1); Helianthus annuus (sunflower) profilin (Hel a 2); Mercurialis annua (annual mercury) profilin (Mer a 1); Cynodon dactylon (Bermuda grass) antigen (Cyn d 1); Cynodon dactylon (Bermuda grass) antigen (Cyn d 7); Cynodon dactylon (Bermuda grass) profilin (Cyn d 12); Dactylis glomerata (orchard grass) AgDg1 antigen (Dac g 1); Dactylis glomerata (orchard grass) antigen (Dac g 2); Dactylis glomerata (orchard grass) antigen (Dac g 3); Dactylis glomerata (orchard grass) antigen (Dac g 5); Holcus lanatus (velvet grass) antigen (Hol 1 1); Lolium perenne (rye grass) group I antigen (Lol p 1); Lolium perenne (rye grass) group II antigen (Lol p 2); Lolium perenne (rye grass) group III antigen (Lol p 3); Lolium perenne (rye grass) group IX antigen (Lol p 5); Lolium perenne (rye grass) antigen (Lol p Ib); Lolium perenne (rye grass) trypsin (Lol p 11); Phalaris aquatica (canary grass) antigen (Pha a 1); Phleum pratense (timothy grass) antigen (Phl p 1); Phleum pratense (timothy grass) antigen (Phl p 2); Phleum pratense (timothy grass) antigen (Phl p 4); Phleum pratense (timothy grass) antigen Ag 25 (Phl p 5); Phleum pratense (timothy grass) antigen (Phl p 6); Phleum pratense (timothy grass) profilin (Phl p 12); Phleum pratense (timothy grass) polygalacturonase (Phl p 13); Poa pratensis (Kentucky blue grass) group I antigen (Poa p 1); Poa pratensis (Kentucky blue grass) antigen (Poa p 5); Sorghum halepense (Johnson grass) antigen (Sor h 1); Alnus glutinosa (alder) antigen (Aln g 1); Betula verrucosa (birch) antigen (Bet v 1); Betula verrucosa (birch) profilin (Bet v 2); Betula verrucosa (birch) antigen (Bet v 3); Betula verrucosa (birch) antigen (Bet v 4); Betula verrucosa (birch) isoflavone reductase homologue (Bet v 5); Betula verrucosa (birch) cyclophilin (Bet v 7); Carpinus betulus (hornbeam) antigen (Car b 1); Castanea sativa (chestnut) Bet v 1 homologue (Cas s 1); Castanea sativa (chestnut) chitinase (Cas s 5); Corylus avelana (hazel) antigen (Cor a 1); Quercus alba (white oak) antigen (Que a 1); Cryptomeria japonica (sugi) antigen (Cry j 1); Cryptomeria japonica (sugi) antigen (Cry j 2); Juniperus ashei (mountain cedar) antigen (Jun a 1); Juniperus ashei (mountain cedar) antigen (Jun a 3); Juniperus oxycedrus (prickly juniper) calmodulin-like antigen (Jun o 2); Juniperus sabinoides (mountain cedar) antigen (Jun s 1); Juniperus virginiana (eastern red cedar) antigen (Jun v 1); Fraxinus excelsior (ash) antigen (Fra e 1); Ligustrum vulgare (privet) antigen (Lig v 1); Olea europea (olive) antigen (Ole e 1); Olea europea (olive) profilin (Ole e 2); Olea europea (olive) antigen (Ole e 3); Olea europea (olive) antigen (Ole e 4); Olea europea (olive) superoxide dismutase (Ole e 5); Olea europea (olive) antigen (Ole e 6); Syringa vulgaris (lilac) antigen (Syr v 1); Acarus siro (mite) fatty acid-binding protein (Aca s 13); Blomia tropicalis (mite) antigen (Blo t 5); Blomia tropicalis (mite) Btlla antigen (Blo t 12); Blomia tropicalis (mite) Bt6 fatty acid-binding protein (Blo t); Dermatophagoides pteronyssinus (mite) antigen P1 (Der p 1); Dermatophagoides pteronyssinus (mite) antigen (Der p 2); Dermatophagoides pteronyssinus (mite) trypsin (Der p 3); Dermatophagoides pteronyssinus (mite) amylase (Der p 4); Dermatophagoides pteronyssinus (mite) antigen (Der p 5); Dermatophagoides pteronyssinus (mite) chymotrypsin (Der p 6); Dermatophagoides pteronyssinus (mite) antigen (Der p 7); Dermatophagoides pteronyssinus (mite) glutathione transferase (Der p 8); Dermatophagoides pteronyssinus (mite) collagenolytic serine prot (Der p 9); Dermatophagoides pteronyssinus (mite) tropomyosin (Der p 10); Dermatophagoides pteronyssinus (mite) apolipophorin like p (Der p 14); Dermatophagoides microceras (mite) antigen (Der m 1); Dermatophagoides farinae (mite) antigen (Der f 1); Dermatophagoides farinae (mite) antigen (Der f 2); Dermatophagoides farinae (mite) antigen (Der f 3); Dermatophagoides farinae (mite) tropomyosin (Der f 10); Dermatophagoides farinae (mite) paramyosin (Der f 11); Dermatophagoides farinae (mite) Mag 3, apolipophorin (Der f 14); Euroglyphus maynei (mite) apolipophorin (Eur m 14); Lepidoglyphus destructor (storage mite) antigen (Lep d 2.0101); Lepidoglyphus destructor (storage mite) antigen (Lep d 2.0102); Bos domesticus (cow) Ag3, lipocalin (Bos d 2); Bos domesticus (cow) alpha-lactalbumin (Bos d 4); Bos domesticus (cow) beta-lactalbumin (Bos d 5); Bos domesticus (cow) serum albumin (Bos d 6); Bos domesticus (cow) immunoglobulin (Bos d 7); Bos domesticus (cow) casein (Bos d 8); Canis familiaris (dog) antigen (Can f 1); Canis familiaris (dog) antigen (Can f 2); Canis familiaris (dog) albumin (Can f?); Equus caballus (horse) lipocalin (Equ c 1); Equus caballus (horse) lipocalin (Equ c 2); Felis domesticus (cat) cat-1 antigen (Fel d 1); Mus musculus (mouse) MUP antigen (Mus m 1); Rattus norvegius (rat) antigen (Rat n 1); Alternaria alternata (fungus) antigen (Alt a 1); Alternaria alternata (fungus) antigen (Alt a 2); Alternaria alternata (fungus) heat shock protein (Alt a 3); Alternaria alternata (fungus) ribosomal protein (Alt a 6); Alternaria alternata (fungus) YCP4 protein (Alt a 7); Alternaria alternata (fungus) aldehyde dehydrogenase (Alt a 10); Alternaria alternata (fungus) enloase (Alt a 11); Alternaria alternata (fungus) acid ribosomal protein P1 (Alt a 12); Cladosporium herbarum (fungus) antigen (Cla h 1); Cladosporium herbarum (fungus) antigen (Cla h 2); Cladosporium herbarum (fungus) aldehyde dehydrogenase (Cla h 3); Cladosporium herbarum (fungus) ribosomal protein); Cladosporium herbarum (fungus) YCP4 protein (Cla h 5); Cladosporium herbarum (fungus) enolase (Cla h 6); Cladosporium herbarum (fungus) acid ribosomal protein P1 (Cla h 12); Aspergillus flavus (fungus) alkaline serine proteinase (Asp fl 13); Aspergillus Fumigatus (fungus) antigen (Asp f 1); Aspergillus Fumigatus (fungus) antigen (Asp f 2); Aspergillus Fumigatus (fungus) peroxisomal protein (Asp f 3); Aspergillus Fumigatus (fungus) antigen (Asp f 4); Aspergillus Fumigatus (fungus) metalloprotease (Asp f 5); Aspergillus Fumigatus (fungus) Mn superoxide dismutase (Asp f 6); Aspergillus Fumigatus (fungus) antigen (Asp f 7); Aspergillus Fumigatus (fungus) ribosomal protein P2 (Asp f 8); Aspergillus Fumigatus (fungus) antigen (Asp f 9); Aspergillus Fumigatus (fungus) aspartis protease (Asp f 10); Aspergillus Fumigatus (fungus) peptidyl-prolyl isomerase (Asp f 11); Aspergillus Fumigatus (fungus) heat shock protein P70 (Asp f 12); Aspergillus Fumigatus (fungus) alkaline serine proteinase (Asp f 13); Aspergillus Fumigatus (fungus) antigen (Asp f 15); Aspergillus Fumigatus (fungus) antigen (Asp f 16); Aspergillus Fumigatus (fungus) antigen (Asp f 17); Aspergillus Fumigatus (fungus) vacuolar serine (Asp f 18); Aspergillus niger (fungus) beta-xylosidase (Asp n 14); Aspergillus niger (fungus) antigen (Asp n 18); Aspergillus niger (fungus) vacuolar serine proteinase; Aspergillus oryzae (fungus) TAKA-amylase A (Asp o 2); Aspergillus oryzae (fungus) alkaline serine proteinase (Asp o 13); Penicillium brevicompactum (fungus) alkaline serine proteinase (Pen b 13); Penicillium citrinum (fungus) heat shock protein P70 (Pen c 1); Penicillium citrinum (fungus) peroxisomal membrane protein (Pen c 3); Penicillium citrinum (fungus) alkaline serine proteinase (Pen c 13); Penicillium notatum (fungus)N-acetyl glucosaminidase (Pen n 1); Penicillium notatum (fungus) alkaline serine proteinase (Pen n 13); Penicillium notatum (fungus) vacuolar serine proteinase (Pen n 18); Penicillium oxalicum (fungus) vacuolar serine proteinase (Pen o 18); Trichophyton rubrum (fungus) antigen (Tri r 2); Trichophyton rubrum (fungus) serine protease (Tri r 4); Trichophyton tonsurans (fungus) antigen (Tri t 1); Trichophyton tonsurans (fungus) serine protease (Tri t 4); Candida albicans (fungus) antigen (Cand a 1); Candida boidinii (fungus) antigen (Cand b 2); Malassezia furfur (fungus) antigen (Mal f 1); Malassezia furfur (fungus) MF1 peroxisomal membrane protein (Mal f 2); Malassezia furfur (fungus) MF2 peroxisomal membrane protein (Mal f 3); Malassezia furfur (fungus) antigen (Mal f 4); Malassezia furfur (fungus) antigen (Mal f 5); Malassezia furfur (fungus) cyclophilin homologue (Mal f 6); Psilocybe cubensis (fungus) antigen (Psi c 1); Psilocybe cubensis (fungus) cyclophilin (Psi c 2); Coprinus comatus (shaggy cap) antigen (Cop c 1); Coprinus comatus (shaggy cap) antigen (Cop c 2); Coprinus comatus (shaggy cap) antigen (Cop c 3); Coprinus comatus (shaggy cap) antigen (Cop c 5); Coprinus comatus (shaggy cap) antigen (Cop c 7); Aedes aegyptii (mosquito) apyrase (Aed a 1); Aedes aegyptii (mosquito) antigen (Aed a 2); Apis mellifera (honey bee) phospholipase A2 (Api m 1); Apis mellifera (honey bee) hyaluronidase (Api m 2); Apis mellifera (honey bee) melittin (Api m 4); Apis mellifera (honey bee) antigen (Api m 6); Bombus pennsylvanicus (bumble bee) phospholipase (Bom p 1); Bombus pennsylvanicus (bumble bee) protease (Bom p 4); Blattella germanica (German cockroach) Bd90k (Bla g 1); Blattella germanica (German cockroach) aspartic protease (Bla g 2); Blattella germanica (German cockroach) calycin (Bla g 4); Blattella germanica (German cockroach) glutathione transferase (Bla g 5); Blattella germanica (German cockroach) troponin C (Bla g 6); Periplaneta americana (American cockroach) Cr-PII (Per a 1); Periplaneta americana (American cockroach) Cr-PI (Per a 3); Periplaneta americana (American cockroach) tropomyosin (Per a 7); Chironomus thummi thummi (midge) hemoglobin (Chi t 1-9); Chironomus thummi thummi (midge) component III (Chi t 1.01); Chironomus thummi thummi (midge) component IV (Chi t 1.02); Chironomus thummi thummi (midge) component I (Chi t 2.0101); Chironomus thummi thummi (midge) component IA (Chi t 2.0102); Chironomus thummi thummi (midge) component II-beta (Chi t 3); Chironomus thummi thummi (midge) component IIIA (Chi t 4); Chironomus thummi thummi (midge) component VI (Chi t 5); Chironomus thummi thummi (midge) component VIIA (Chi t 6.01); Chironomus thummi thummi (midge) component IX (Chi t 6.02); Chironomus thummi thummi (midge) component VIIB (Chi t 7); Chironomus thummi thummi (midge) component VIII (Chi t 8); Chironomus thummi thummi (midge) component X (Chi t 9); Dolichovespula maculata (white face hornet) phospholipase (Dol m 1); Dolichovespula maculata (white face hornet) hyaluronidase (Dol m 2); Dolichovespula maculata (white face hornet) antigen 5 (Dol m 5); Dolichovespula arenaria (yellow hornet) antigen 5 (Dol a 5); Polistes annularies (wasp) phospholipase A1 (Pol a 1); Polistes annularies (wasp) hyaluronidase (Pol a 2); Polistes annularies (wasp) antigen 5 (Pol a 5); Polistes dominulus (Mediterranean paper wasp) antigen (Pol d 1); Polistes dominulus (Mediterranean paper wasp) serine protease (Pol d 4); Polistes dominulus (Mediterranean paper wasp) antigen (Pol d 5); Polistes exclamans (wasp) phospholipase A1 (Pol e 1); Polistes exclamans (wasp) antigen 5 (Pol e 5); Polistes fuscatus (wasp) antigen 5 (Pol f 5); Polistes metricus (wasp) antigen 5 (Pol m 5); Vespa crabo (European hornet) phospholipase (Vesp c 1); Vespa crabo (European hornet) antigen 5 (Vesp c 5.0101); Vespa crabo (European hornet) antigen 5 (Vesp c 5.0102); Vespa mandarina (giant Asian hornet) antigen (Vesp m 1.01); Vespa mandarina (giant Asian hornet) antigen (Vesp m 1.02); Vespa mandarina (giant Asian hornet) antigen (Vesp m 5); Vespula flavopilosa (yellowjacket) antigen 5 (Ves f 5); Vespula germanica (yellowjacket) antigen 5 (Ves g 5); Vespula maculifrons (yellowjacket) phospholipase A1 (Ves m 1); Vespula maculifrons (yellowjacket) hyaluronidase (Ves m 2); Vespula maculifrons (yellowjacket) antigen 5 (Ves m 5); Vespula pennsylvanica (yellowjacket) (antigen 5Ves p 5); Vespula squamosa (yellowjacket) antigen 5 (Ves s 5); Vespula vidua (wasp) antigen (Ves vi 5); Vespula vulgaris (yellowjacket) phospholipase A1 (Ves v 1); Vespula vulgaris (yellowjacket) hyaluronidase (Ves v 2); Vespula vulgaris (yellowjacket) antigen 5 (Ves v 5); Myrmecia pilosula (Australian jumper ant) antigen (Myr p 1); Myrmecia pilosula (Australian jumper ant) antigen (Myr p 2); Solenopsis geminata (tropical fire ant) antigen (Sol g 2); Solenopsis geminata (tropical fire ant) antigen (Sol g 4); Solenopsis invicta (fire ant) antigen (Sol i 2); Solenopsis invicta (fire ant) antigen (Sol i 3); Solenopsis invicta (fire ant) antigen (Sol i 4); Solenopsis saevissima (Brazilian fire ant) antigen (Sol s 2); Gadus callarias (cod) allergen M (Gad c 1); Salmo salar (Atlantic salmon) parvalbumin (Sal s 1); Gallus domesticus (chicken) ovomucoid (Gal d 1); Gallus domesticus (chicken) ovalbumin (Gal d 2); Gallus domesticus (chicken) conalbumin; A22 (Gal d 3); Gallus domesticus (chicken) lysozyme (Gal d 4); Gallus domesticus (chicken) serum albumin (Gal d 5); Metapenaeus ensis (shrimp) tropomyosin (Met e 1); Penaeus aztecus (shrimp) tropomyosin (Pen a 1); Penaeus indicus (shrimp) tropomyosin (Pen i 1); Todarodes pacificus (squid) tropomyosin (Tod p 1); Haliotis Midae (abalone) antigen (Hal m 1); Apium graveolens (celery) Bet v 1 homologue (Api g 1); Apium graveolens (celery) profilin (Api g 4); Apium graveolens (celery) antigen (Api g 5); Brassica juncea (oriental mustard) 2S albumin (Bra j 1); Brassica rapa (turnip) prohevein-like protein (Bar r 2); Hordeum vulgare (barley) BMAI-1 (Hor v 1); Zea mays (maize, corn) lipid transfer protein (Zea m 14); Corylus avellana (hazelnut) Bet v 1 homologue (Cor a 1.0401); Malus domestica (apple) Bet v 1 homologue (Mal d 1); Malus domestica (apple) lipid transfer protein (Mal d 3); Pyrus communis (pear) Bet v 1 homologue (Pyr c 1); Pyrus communis (pear) profilin (Pyr c 4); Pyrus communis (pear) isoflavone reductase homologue (Pyr c 5); Oryza sativa (rice) antigen (Ory s 1); Persea americana (avocado) endochitinase (Pers a 1); Prunus armeniaca (apricot) Bet v 1 homologue (Pru ar 1); Prunus armeniaca (apricot) lipid transfer protein (Pru ar 3); Prunus avium (sweet cherry) Bet v 1 homologue (Pru av 1); Prunus avium (sweet cherry) thaumatin homologue (Pru av 2); Prunus avium (sweet cherry) profilin (Pru av 4); Prunus persica (peach) lipid transfer protein (Pru p 3); Sinapis alba (yellow mustard) 2S albumin (Sin a 1); Glycine max (soybean) HPS (Gly m 1.0101); Glycine max (soybean) HPS (Gly m 1.0102); Glycine max (soybean) antigen (Gly m 2); Glycine max (soybean) profilin (Gly m 3); Arachis hypogaea (peanut) vicilin (Ar a h 1); Arachis hypogaea (peanut) (conglutin Ar a h 2); Arachis hypogaea (peanut) glycinin (Ar a h 3); Arachis hypogaea (peanut) glycinin (Ar a h 4); Arachis hypogaea (peanut) (profilin Ar a h 5); Arachis hypogaea (peanut) conglutin homologue (Ar a h 6); Arachis hypogaea (peanut) conglutin homologue (Ar a h 7); Actinidia chinensis (kiwi) cysteine protease (Act c 1); Solanum tuberosum (potato) patatin (Sol t 1); Bertholletia excelsa (Brazil nut) 2S albumin (Ber e 1); Juglans regia (English walnut) 2S albumin (Jug r 1); Juglans regia (English walnut) vicilin (Jug r 2); Ricinus communis (castor bean) 2S albumin (Ric c 1); Anisakis simplex (nematode) antigen (Ani s 1); Anisakis simplex (nematode) paramyosin (Ani s 2); Ascaris suum (worm) antigen (Asc s 1); Aedes aegyptii (mosquito) apyrase (Aed a 1); Aedes aegyptii (mosquito) antigen (Aed a 2); Hevea brasiliensis (rubber) elongation factor (Hev b 1); Hevea brasiliensis (rubber) 1,3-glucanase (Hev b 2); Hevea brasiliensis (rubber) antigen (Hev b 3); Hevea brasiliensis (rubber) component of microhelix protein complex (Hev b 4); Hevea brasiliensis (rubber) antigen (Hev b 5); Hevea brasiliensis (rubber) hevein precursor (Hev b 6.01); Hevea brasiliensis (rubber) hevein (Hev b 6.02); Hevea brasiliensis (rubber)C-terminal fragment antigen (Hev b 6.03); Hevea brasiliensis (rubber) patatin homologue (Hev b 7); Hevea brasiliensis (rubber) profilin (Hev b 8); Hevea brasiliensis (rubber) enolase (Hev b 9); Hevea brasiliensis (rubber) Mn-superoxide dismut (Hev b 10); and Ctenocephalides felis felis (cat flea) antigen (Cte f 1). Preferred targets include food allergens such as from nuts, fish, milk, etc., as well as venoms, pollens, dander, latex, fungi, medicines (including antibiotics) and in particular peanut, milk, shellfish, tree nuts, egg, fin fish, wheat, soy, and sesame.

1-23. (canceled)

24. A method of providing a pharmaceutical composition for treatment of an allergy to an allergen, comprising:
identifying a first epitope bin and a second epitope bin of the allergen that each include antibodies that have greater therapeutic efficacies relative to antibodies from other epitope bins of the allergen;
selecting a first antibody from the first epitope bin;
selecting a second antibody from the second epitope bin; and
formulating the first antibody and the second antibody to provide the pharmaceutical composition.

25. The method of claim 24, further comprising, prior to identifying the first epitope bin and the second epitope bin, comparing the therapeutic efficacies of antibodies from the epitope bins of the allergen by assessing relative abilities of the antibodies from the epitope bins to inhibit IgE binding to the allergen, wherein the first epitope bin and the second epitope bin each include antibodies showing greater inhibition of IgE binding to the allergen relative to antibodies from the other epitope bins of the allergen.

26. The method of claim 24, further comprising, prior to identifying the first epitope bin and the second epitope bin, comparing the therapeutic efficacies of antibodies from the epitope bins of the allergen by assessing relative abilities of the antibodies from the epitope bins to inhibit allergen mediated activation in cells, wherein the first epitope bin and the second epitope bin each include antibodies showing greater inhibition of allergen mediated activation in cells relative to antibodies from the other epitope bins of the allergen.

27. The method of claim 26, wherein the relative abilities of the antibodies from the epitope bins to inhibit allergen mediated activation in cells is assessed using a basophil activation test (BAT).

28. The method of claim 26, wherein the relative abilities of the antibodies from the epitope bins to inhibit allergen mediated activation in cells is assessed using a mast cell activation test (MAT).

29. The method of claim 24, wherein the allergen is an allergen originating from the group consisting of: peanut, milk, egg, tree nut, fish, shellfish, soy, legume, seed, and wheat.

30. The method of claim 24, wherein the allergen is a peanut allergen.

31. The method of claim 30, wherein the peanut allergen is Ara h 2.

32. The method of claim 30, wherein the peanut allergen is Ara h 6.

33. The method of claim 24, wherein the allergen is Fel d 1.

34. The method of claim 24, wherein the first antibody and the second antibody are monoclonal antibodies.

35. The method of claim 24, wherein the first antibody and/or the second antibody are IgG antibodies.

36. The method of claim 24, wherein the first antibody and/or the second antibody are monoclonal IgG4 antibodies.

37. The method of claim 24, wherein the first antibody binds to a first epitope and the second antibody binds to a second epitope, and wherein the first epitope and the second epitope are selected for minimal cross-reactivity with the second antibody and the first antibody, respectively.

38. The method of claim 24, further comprising formulating the pharmaceutical composition with an anti-IgE antibody in an amount sufficient to reduce, but not eliminate, circulating IgE.

39. A method of providing a pharmaceutical composition for treatment of an allergy to an allergen, comprising:
selecting a first antibody from a first epitope bin of the allergen;
selecting a second antibody from a second epitope bin of the allergen, wherein the first antibody and the second antibody do not block each other from binding to the allergen, and wherein the first epitope bin and the second epitope bin include antibodies showing greater inhibition of allergen mediated activation in cells relative to antibodies from other epitope bins of the allergen; and
combining the first antibody and the second antibody to provide the pharmaceutical composition.

40. A method of providing a pharmaceutical composition for treatment of an allergy to an allergen, comprising:
identifying epitope bins of the allergen, wherein each epitope bin includes antibodies that block one another from binding to the allergen;
comparing abilities of antibodies from the epitope bins of the allergen to inhibit allergen mediated activation in cells;
selecting a first epitope bin and a second epitope bin from the epitope bins, wherein the first epitope bin and the second epitope bin include antibodies showing higher inhibition of allergen mediated activation in cells relative to antibodies from other epitope bins of the allergen;
selecting a first antibody from the first epitope bin;
selecting a second antibody from the second epitope bin; and
formulating the first antibody and the second antibody to provide the pharmaceutical composition.

41. The method of claim 40, wherein the first antibody and/or the second antibody are monoclonal IgG4 antibodies.

42. The method of claim 40, wherein the allergen is a peanut allergen or Fel d 1.

43. The method of claim 40, wherein the abilities of the antibodies from the epitope bins of the allergen to inhibit allergen mediated activation in cells are compared using a basophil activation test (BAT) or a mast cell activation test (MAT).
